(19) United States
(12) Patent Application Publication
(10) Pub. No.: US 2023/0355533 A1
(43) Pub. Date: Nov. 9, 2023

(54) BIODEGRADABLE POLYCAPROLACTONE CAPSULE FOR ANTIBODY RELEASE AND DEGRADATION

(71) Applicant: University Of Cincinnati, Cincinnati, OH (US)

(72) Inventors: Yoonjee Park, Montgomery, OH (US); Thomas Waterkotte, Cincinnati, OH (US)

(21) Appl. No.: 18/313,754

(22) Filed: May 8, 2023

Related U.S. Application Data
(60) Provisional application No. 63/339,395, filed on May 6, 2022.

(51) Int. Cl. A61K 9/48; C07K 16/22; A61K 31/573; A61J 3/07

(52) U.S. Cl. CPC A61K 9/4808; A61K 9/4816; C07K 16/22; A61K 31/573; A61K 9/4858; A61J 3/07; A61K 9/4833; C07K 2317/24

(57) ABSTRACT

A biodegradable capsule implant is provided. The biodegradable capsule includes a capsule body having an inner surface and an outer surface. The capsule body includes a polymer, and the inner surface defines a hollow interior space. The biodegradable capsule further includes a therapeutic agent housed in the hollow interior space. The biodegradable capsule further includes a plurality of pores on the capsule body. The capsule body is configured to absorb the therapeutic agent at the inner surface and desorb the therapeutic agent out of the outer surface.

### CROSS REFERENCE TO RELATED APPLICATIONS

The present application is a United States Non-Provisional Patent Application which claims priority to, and the benefit of the filing date of, U.S. Ser. No. 63/339395, titled “Biodegradable Polycaprolactone Capsule For Antibody Release and Degradation”, which was filed May 8, 2022 the disclosure of which is hereby incorporated herein by reference in its entirety.

### FIELD OF THE INVENTION

This invention relates generally to biodegradable implants, and more particularly to biodegradable polycaprolactone capsules.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Monoclonal antibodies (mAbs) are the largest class of biopharmaceuticals and are used in the treatment of many chronic diseases including cancer, immunological disorders, and infectious diseases. US Food and Drug Administration (FDA) and European Medicines Agency (EMA) approved 36 mAb drugs from 2013 to 2017, and the FDA approved 23 new mAbs in 2018-2020. The size of the global mAbs market was worth USD 39.1 billion in 2021 and this figure is estimated to be growing and will reach USD 50.62 billion by 2026. This is because the number of patients treated by mAbs is increasing as well as the possibility of more mAbs coming to market is rising.

Therapeutic mAbs have been widely developed and used for chronic disease treatment. For example, bevacizumab or ranibizumab have been used for chronic age-related macular degeneration (AMD) or various types of cancer. Treatment of the diseases involves the suppression of the vascular endothelial growth factor (VEGF) protein. Current standard of care for AMD is repetitive monthly intravitreal injections of these mAbs directly to the eye. The frequent injections are necessary partly because of a relatively short half-life of the drugs in the target sites. Bevacizumab (Bev) administered intravitreally has a half-life of 5 to 10 days. However, the frequent intravitreal injections can cause issues, including hemorrhage, corneal erosion, and post-injection ocular surface discomfort (i.e. burning, stinging, or itching). In addition, this is a significant burden to the patient, the patient's family, and the healthcare system.

There is no biodegradable drug delivery system for long-term sustained antibody release (i.e. over a month) to date. Although a port delivery device has been recently launched for delivery of ranibizumab for posterior eye disease treatment, it is not biodegradable and may require additional surgery for removal. The port delivery system is surgically inserted via the pars plana area in the superotemporal quadrant of the eye and releases antibodies sustainably for several months. However, long-term durability and whether it should be replaced after a certain duration of time, as well as the appropriate procedure for safe removal of the implant should complications arise are uncertain.

Furthermore, although there are biodegradable implants available for small drug molecules, such as steroids or hormones, it has been challenging to develop a system that releases antibodies which are much bigger than typical drug molecules with several hundred Da, for example ˜150 kDa. The typical method of implant fabrication is a melt-extrusion method, which requires high temperature (100˜200° C.) to melt polymers. Some drug molecules are stable at the high temperature and can be mixed with the polymer to be a matrix-type implant. However, antibodies are not stable at the high temperature and this fabrication method cannot be used for antibody implant fabrication.

Although therapy using monoclonal antibodies (mAbs) has been steadily successful over the last 20 years, the means of delivery of mAbs has not been optimized, especially for long-term delivery. Frequent injections or infusions have been the current standard of care. These forms of treatment may be painful, invasive, or otherwise inconvenient. Accordingly, improvements to monoclonal antibody delivery methods are needed.

Dexamethasone, one of the most widely used corticosteroids, is an effective anti-inflammatory drug commonly prescribed for chronic inflammatory diseases, such as asthma, allergic rhinitis, urticaria, and many other conditions. Dexamethasone (Dex) has been also employed to prevent and/or reduce vocal fold scarring because of the effect on wound healing, improving post operative voice quality in patients undergoing phonosurgery. However, the use of Dex is limited by dosing. Oral dosing is less effective in target tissues because of the necessary high dose and is limited by serious side effects, including ocular, musculoskeletal, and dermatologic diseases. In addition is the need for frequent injections for local delivery in places challenging to access, such as the larynx. Local injections to the vocal folds are limited by the complex three-dimensional laryngeal anatomy and lack of easy access.

Due to the short half-life of Dex being 5.5 h, many studies have been focused on exploring a drug delivery system for its long-term sustained release. One successful example is the intravitreal Dex implant (Ozurdex®, Allergan® Inc, Irvine, CA), which is a rod-shaped implant made of solid biodegradable poly(lactide-co-glycolide) (PLGA) polymer. This Dex implant is designed to release 700 tg of Dex over six months with a peak concentration at day 22, and was approved by the Food and Drug Administration (FDA) for the treatment of macular edema in 2009 with minimum side effects. However, most of the drug is released within 1-2 months. PLGA used in Ozurdex® is a polyester-based polymer, which is the most widely investigated for drug delivery. Other polyester-based polymers include polycaprolactone (PCL), poly(lactic acid) (PLA), and poly(glycolic acid) (PGA). Many drug delivery systems have been explored based on design and synthesis of combinations of these polymers.

Several studies demonstrated therapeutic delivery of growth factors or extracellular matrix (ECM) compositions to the vocal fold utilizing biocompatible polymers such as PCL, with no significant immune or foreign body responses. In addition, one study has shown that a surgical micro-clip made of magnesium for laryngeal microsurgery suppressed inflammation when the surface was coated with PCL. These studies indicate that biocompatible polymers are very promising tools for the delivery of desired treatment in the vocal fold.

However, it is nontrivial to select a drug delivery carrier among the polymer options because of miscibility between a drug and a polymer, a drug release kinetics, and safety against immune responses and toxicity.

### SUMMARY OF THE INVENTION

Aspects of the disclosed invention are directed to multimode sensors integrated onto a feature to form a biodegradable capsule implant. Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

A biodegradable capsule implant is provided. The biodegradable capsule includes a capsule body having an inner surface and an outer surface. The capsule body includes a polymer, and the inner surface defines a hollow interior space. The biodegradable capsule further includes a therapeutic agent housed in the hollow interior space. The biodegradable capsule further includes a plurality of pores on the capsule body. The capsule body is configured to absorb the therapeutic agent at the inner surface and desorb the therapeutic agent out of the outer surface.

In a related embodiment, the polymer includes an aliphatic polyester selected from a group consisting of polycaprolactone (PCL), poly(lactic acid) (PLA), 90:10 poly(lactic-co-glycolide) (PLGA), and 50:50 PLGA, and combinations thereof.

In a related embodiment, wherein the polymer includes polycaprolactone (PCL).

In a related embodiment, the therapeutic agent includes either an antibody or an anti-inflammatory agent.

In a related embodiment, the therapeutic agent includes the antibody.

In a related embodiment, the antibody includes Bevacizumab or ranibizumab.

In a related embodiment, the therapeutic agent includes the anti-inflammatory agent.

In a related embodiment, the anti-inflammatory agent includes dexamethasone.

In a related embodiment, the anti-inflammatory agent has a therapeutic effect when treating tumor necrosis factor alpha-induced inflammation.

In a related embodiment, a diameter of the plurality of pores is greater than a diameter of the therapeutic agent.

In a related embodiment, each of the plurality of pores has a diameter less than 1 micron in length.

In a related embodiment, the capsule body further includes polyethylene glycol.

In a related embodiment, the capsule body includes the polyethylene glycol and the polymer in a weight ratio of between more than 0.0 and less than or equal to 0.1 of the polyethylene glycol to the polymer.

In a related embodiment, the therapeutic agent is a first therapeutic agent, and the biodegradable capsule implant further includes a second therapeutic agent.

In a related embodiment, the first therapeutic agent is Bevacizumab and the second therapeutic agent is ranibizumab.

A method of supplying a therapeutic agent to a recipient is also provided. The method includes providing a biodegradable capsule implant including a capsule body. The capsule body includes a polymer, and the capsule body has an inner surface and an outer surface, wherein the inner surface defines a hollow interior space. The biodegradable capsule implant further includes a therapeutic agent housed in the hollow interior space. The biodegradable capsule implant further includes a plurality of pores on the capsule body, and the capsule body is configured to absorb the therapeutic agent at the inner surface and desorb the therapeutic agent out of the outer surface. The biodegradable capsule is configured to release the therapeutic agent for a predetermined amount of time when the biodegradable capsule implant is administered to the recipient.

In a related embodiment, the predetermined amount of time is at least 50 days.

In a related embodiment, the predetermined amount of time is at least 200 days.

A method of making a biodegradable capsule implant is also provided. The method includes stacking a plurality of polymer sheets onto each other. The method further includes rolling the plurality of polymer sheets, thus stacked, circumferentially to form a generally cylindrical shape defining a hollow cylindrical space, the hollow cylindrical space being configured to receive a therapeutic agent therein, the generally cylindrical shape comprising a first open end and a second open end. The method further includes submerging the generally cylindrical shape into a deionized water bath to form a plurality of pores through the generally cylindrical shape. The method further includes depositing the therapeutic agent in the generally cylindrical space via either the first open end or the second open end. The method further includes sealing the first open end and the second open end to form the biodegradable capsule implant.

A method of making a biodegradable capsule implant is also provided. The method includes stacking a plurality of polymer sheets onto each other. The method further includes rolling the plurality of polymer sheets, thus stacked, circumferentially to form a generally cylindrical shape defining a hollow cylindrical space, the hollow cylindrical space being configured to receive a therapeutic agent therein, the generally cylindrical shape comprising a first open end and a second open end. The method further includes sonicating the generally cylindrical shape to form a plurality of pores through the generally cylindrical shape. The method further includes depositing the therapeutic agent in the generally cylindrical space via either the first open end or the second open end. The method further includes sealing the first open end and the second open end to form the biodegradable capsule implant.

### BRIEF DESCRIPTION OF THE DRAWINGS

The objects and advantages of the disclosed invention will be further appreciated in light of the following detailed descriptions and drawings in which:

FIG. 1 is a schematic of an embodiment of a nano-porous PCL antibody implant according to the invention.

FIG. 2 is a series of images showing one embodiment of the implant fabrication process of the invention.

FIG. 3A an SEM image of a 0.05 PEG PCL overall (scale bar 500 μm) according to an embodiment of the invention.

FIG. 3B an SEM image of a 0.05 PEG PCL sheet top according to an embodiment of the invention. Scale bars=10 μm

FIG. 3C an SEM image of a 0.05 PEG PCL sheet bottom according to an embodiment of the invention. Scale bars=10 μm

FIG. 3D is a cross-section of a 0.0 PEG PCL according to an embodiment of the invention. Scale bars=10 μm

FIG. 3E is a cross-section of a 0.05 PEG PCL according to an embodiment of the invention. Scale bars=10 μm

FIG. 3F is a cross-section of a 0.05 PEG PCL according to an embodiment of the invention. Scale bars=10 μm.

FIG. 4 is a graph showing drug release profile in natural condition.

FIG. 5 is a graph showing accelerated drug release.

FIG. 6 is a graph showing NaOH vs polybutylene succinate (PBS) Time for release.

FIG. 7A is a cross-sectional SEM image of a 0.05 PEG capsule after 2 months of natural degradation. Scale bars=10 μm for cross-section and 500 μm for overall images.

FIG. 7B is an overall SEM image of a 0.05 PEG capsule after 2 months of natural degradation. Scale bars=10 μm for cross-section and 500 μm for overall images.

FIG. 7C is a cross-sectional SEM image of a 0.05 PEG capsule after 4 months of natural degradation. Scale bars=10 μm for cross-section and 500 μm for overall images.

FIG. 7D is an overall SEM image of a 0.05 PEG capsule after 4 months of natural degradation. Scale bars=10 μm for cross-section and 500 μm for overall images.

FIG. 7E is a cross-sectional SEM image of a 0.05 PEG capsule after 6 months of natural degradation. Scale bars=10 μm for cross-section and 500 μm for overall images.

FIG. 7F is an overall SEM image of a 0.05 PEG capsule after 6 months of natural degradation. Scale bars=10 μm for cross-section and 500 μm for overall images.

FIG. 8A is an overall SEM image of a 0.0 PEG capsule after 4 days of accelerated degradation. Scale bars=500 μm for all images.

FIG. 8B is an overall SEM image of a 0.0 PEG capsule after 10 days of accelerated degradation. Scale bars=500 μm for all images.

FIG. 8C is an overall SEM image of a 0.0 PEG capsule after 20 days of accelerated degradation. Scale bars=500 μm for all images.

FIG. 8D is an overall SEM image of a 0.0 PEG capsule after 30 days of accelerated degradation. Scale bars=500 μm for all images.

FIG. 8E is an overall SEM image of a 0.05 PEG capsule after 4 days of accelerated degradation. Scale bars=500 μm for all images.

FIG. 8F is an overall SEM image of a 0.05 PEG capsule after 10 days of accelerated degradation. Scale bars=500 μm for all images.

FIG. 8G is an overall SEM image of a 0.05 PEG capsule after 20 days of accelerated degradation. Scale bars=500 μm for all images.

FIG. 8H is an overall SEM image of a 0.05 PEG capsule after 30 days of accelerated degradation. Scale bars=500 μm for all images.

FIG. 8I is an overall SEM image of a 0.1 PEG capsule after 4 days of accelerated degradation. Scale bars=500 μm for all images.

FIG. 8J is an overall SEM image of a 0.1 PEG capsule after 10 days of accelerated degradation. Scale bars=500 μm for all images.

FIG. 8K is an overall SEM image of a 0.1 PEG capsule after 20 days of accelerated degradation. Scale bars=500 μm for all images.

FIG. 8L is an overall SEM image of a 0.1 PEG capsule after 30 days of accelerated degradation. Scale bars=500 μm for all images.

FIG. 9A is a cross-sectional SEM image of a 0.0 PEG capsule after 4 days of accelerated degradation. Scale bars=10 μm for all images.

FIG. 9B is a cross-sectional SEM image of a 0.0 PEG capsule after 10 days of accelerated degradation. Scale bars=10 μm for all images.

FIG. 9C is a cross-sectional SEM image of a 0.0 PEG capsule after 20 days of accelerated degradation. Scale bars=10 μm for all images.

FIG. 9D is a cross-sectional SEM image of a 0.0 PEG capsule after 30 days of accelerated degradation. Scale bars=10 μm for all images.

FIG. 9E is a cross-sectional SEM image of a 0.05 PEG capsule after 4 days of accelerated degradation. Scale bars=10 μm for all images.

FIG. 9F is a cross-sectional SEM image of a 0.05 PEG capsule after 10 days of accelerated degradation. Scale bars=10 μm for all images.

FIG. 9G is a cross-sectional SEM image of a 0.05 PEG capsule after 20 days of accelerated degradation. Scale bars=10 μm for all images.

FIG. 9H is a cross-sectional SEM image of a 0.05 PEG capsule after 30 days of accelerated degradation. Scale bars=10 μm for all images.

FIG. 9I is a cross-sectional SEM image of a 0.1 PEG capsule after 4 days of accelerated degradation. Scale bars=10 μm for all images.

FIG. 9J is a cross-sectional SEM image of a 0.1 PEG capsule after 10 days of accelerated degradation. Scale bars=10 μm for all images.

FIG. 9K is a cross-sectional SEM image of a 0.1 PEG capsule after 20 days of accelerated degradation. Scale bars=10 μm for all images.

FIG. 9L is a cross-sectional SEM image of a 0.1 PEG capsule after 30 days of accelerated degradation. Scale bars=10 μm for all images.

FIG. 10 are bar graphs showing implant characteristics over time in natural and accelerated conditions.

FIG. 11 is a graph showing permeability data for cumulative drug permeation vs. time.

FIG. 12 is a graph showing a model fitting for 6 months.

FIG. 13A is an SEM image of a sheet of 50:50 PLGA.

FIG. 13B is an SEM image of a sheet of 90:10 PLGA.

FIG. 13C is an SEM image of a sheet of PLA.

FIG. 13D is an SEM image of a sheet of PCL.

FIG. 13E is a cross-sectional image of a sheet of 50:50 PLGA.

FIG. 13F is a cross-sectional image of a sheet of 90:10 PLGA.

FIG. 13G is a cross-sectional image of a sheet of PLA.

FIG. 13H is a cross-sectional image of a sheet of PCL.

FIG. 14 is a plot of Dex release profile in percentage vs. time for 50:50 PLGA, PLA, 90:10 PLGA, and PCL.

FIG. 15 is a plot of cumulative drug permeation vs. time for 50:50 PLGA, PLA, 90:10 PLGA, and PCL.

FIG. 16 is a bar graph showing IL-6-ELISA assay using TNF-α stimulated HUVECs—#p<0.05, and **p<0.01.

FIG. 17A is a graph of a model fitting for 50:50 PLGA to measured data.

FIG. 17B is a graph of a model fitting for PLA to measured data.

FIG. 17C is a graph of a model fitting for 90:10 PLGA to measured data.

FIG. 17D is a graph of a model fitting for PCL to measured data.

FIG. 18A are representative images of HUVECs for five conditions before adding MTT reagents.

FIG. 18B is a bar graph showing in vitro cytotoxicity results using MTT assay—#p<0.05, and **p<0.01 vs. an example embodiment of the invention.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The present invention involves a nano-porous capsule implant that may be configured to release antibodies slowly over a desired amount of time such as at least 50 days, at least 100 days, or at least 200 days. The nano-porous capsule implants are degradable in the body utilizing polycaprolactone (PCL). PCL is an FDA approved biodegradable polymer for use in implantable biomaterials and injectable implants. The slow degradation and hydrophobicity of PCL is one reason why it is extensively used in long-term systems. In the present invention, PCL is used to develop Bev (antibody) implants for a long-term biodegradable drug delivery platform.

In addition, degradable polymers are known to undergo morphological changes over degradation and these changes may affect the antibody release kinetics from the implant. For examples, pore size in the polymer increases over degradation and surface erosion can reduce the thickness of polymer, which may impact on faster antibody release from the implant. For the present invention, the antibody release kinetics for the long-term and whether or not the kinetics are affected by the degradation are determined. Because the degradation rate of PCL is slow, which takes at least 6 months and up to 4 years for noticeable degradation, the release kinetics and the degradation of the implant are determined in an accelerated condition.

The polymer degradation proceeds via mainly three pathways, i.e. biological, chemical or physical means. In vivo and in vitro degradation were reported to occur at the same rate, suggesting no significant contribution by enzymes initially. Biological degradation was more pronounced when MW (molecular weight) reaches ˜5000 for PCL. The main mode of degradation for high molecular-weight aliphatic polyesters is hydrolytic random scission. Thus, accelerated degradation using an acidic or basic medium, which enhances the hydrolysis of polyester, mimics physiological conditions better than other methods, such as temperature acceleration. A basic medium, such as a sodium hydroxide (NaOH) solution, was widely studied for accelerated polymer hydrolysis/degradation. Although it is suggested by ASTM International (American Society for Testing and Materials) to compare in the morphological structure between natural and accelerated degradation, the accelerated degradation method is useful to determine the drug release kinetics over degradation.

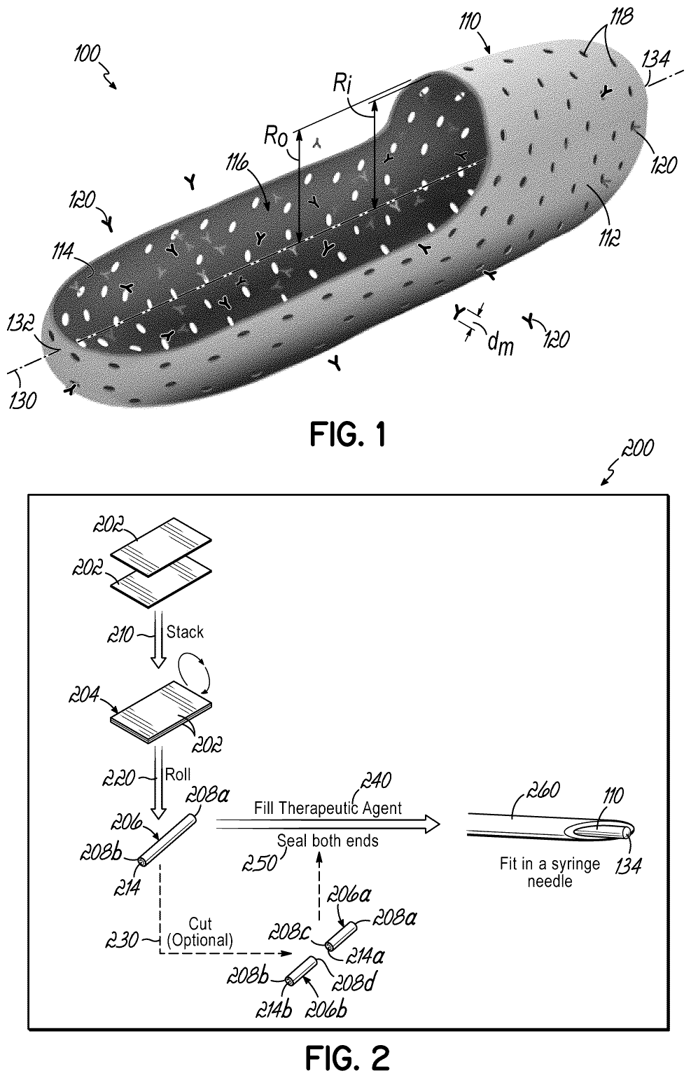

In one embodiment, the present invention uses nano-porous (i.e. submicron) PCL capsule implants developed for long-term antibody release with three different pore sizes and drug release rates in the accelerated degradation conditions for the first time, as shown in the schematics of FIG. 1. Furthermore, the antibody release kinetics are fit to mathematical models using permeability and partition coefficient values determined by experiment. In addition, the effectiveness of the accelerated system for predicting the long-term drug release kinetics was evaluated.

Long-term antibody biodegradable implant using a porous polycaprolactone (PCL) capsule are provided herein. These capsules released bevacizumab (Bev) slowly for 8 months to date. The Bev release kinetics fit a drug release model with experimental data of the diffusion coefficient and partition coefficient through the polymer capsule. Since screening drug release profiles for the long term (>6 months) is time consuming, an accelerated degradation method was used after validating the characteristics of the PCL capsule in natural and accelerated degradation conditions. The correlation of the time period between natural and accelerated degradation was determined. Overall, it has been discovered that mAbs can be released from a porous PCL capsule without an effect of the polymer degradation over a long period (˜6 months) and the long-term release kinetics can be determined by the accelerated degradation within 14 days.

In another embodiment, sustained Dex (dexamethasone) capsule implants for sustained local delivery for inflammatory disease treatment is provided. The capsule implants include a capsule including a polymer. In some examples, the polymer is biodegradable, and is selected from a group consisting of polycaprolactone (PCL), poly(lactic acid) (PLA), 90:10 poly(lactic-co-glycolide) (PLGA), and 50:50 PLGA, and combinations thereof. In embodiments of the invention, at least one of these polymers with different compositions, PCL, PLA, 90:10 PLGA, and 50:50 PLGA, and combinations thereof, are included in a capsule according to the invention, and tested for release of dexamethasone. All of these polymers are aliphatic polyesters, and also FDA-approved in many medical applications.

Drug release profiles from capsules including at least one of each of these four polymers may be compared and fit to a cylindrical reservoir first-order kinetics model. As a result, 50:50 PLGA showed the fastest release with the largest permeability and partition coefficient at 0.4909 nm/s and 1.9519, respectively. On the other hand, PCL showed the slowest release with the smallest permeability and partition coefficient at 0.1915 nm/s and 0.8872, respectively. The results indicate that the drug release kinetics are highly correlated with hydrophobicity of the polymer sheet: the more hydrophobic, the slower the drug release kinetics for the hydrophilic drug. The in vitro therapeutic efficacy of the Dex implant was also explored using Tumor Necrosis Factor Alpha (TNF-α) stimulated human umbilical vein endothelial cells (HUVECs), showing effective suppression of IL-6 levels with the implant compared to free Dex with minimal toxicity. Overall, there is evidence that the release trend of Dex from implants follows the hydrophobicity of each polymer, and the Dex implant inhibits the IL-6 expression effectively.

PCL is a semicrystalline linear polyester which may be obtained by the ring-opening polymerization of epsilon-caprolactone. PLA may be produced through the direct condensation reaction of its monomer lactide and is a hydrophobic polymer due to the CH₃ side groups. PGA may be prepared by ring opening polymerization of a cyclic lactone, glycolide. Due to its excellent mechanical properties but low solubility and high degradation rate against acidic products, PGA may be prepared as copolymers. PLGA is a copolymer of PLA and PGA and different ratios of PLGA have been commercially developed. When the ratio of lactide/glycolide (L/G) increases, the degradation rate of the copolymer decreases and the hydrophobicity increases.

Referring to FIG. 1, a nano-porous capsule implant 100 is provided. The nano-porous capsule implant 100 includes a capsule body 110 having an outer surface 112 and an inner surface 114. The inner surface 114 is shaped to define a hollow interior space 116, and the capsule body 110 includes a plurality of pores 118 thereon. The hollow interior space 116 of the nano-porous capsule implant 100 is configured to house therapeutic agents 120. Each of the plurality of pores 118 are channels that permit the mass transfer of the therapeutic agent 120 housed in the hollow interior space 116 to spaces outside of the hollow interior space 116 defined by the capsule body 110.

In embodiments of the invention, the capsule body 110 includes materials such as a polymer. In some embodiments, one or more polymers included in the capsule body 110 are selected from a group consisting of polycaprolactone (PCL), poly(lactic acid) (PLA), 90:10 poly(lactic-co-glycolide) (PLGA), and 50:50 PLGA, and combinations thereof. Alternatively or in addition, the capsule body 110 may include polyethylene glycol (PEG). In some examples, the capsule body 110 includes no polyethylene glycol. In other examples, polyethylene glycol may be included in the capsule body 110 in a ratio with another polymer, such as the polymer selected from the group consisting of polycaprolactone (PCL), poly(lactic acid) (PLA), 90:10 poly(lactic-co-glycolide) (PLGA), and 50:50 PLGA, and combinations thereof. A weight ratio of PEG to another polymer included in the capsule body 110 may be between 0.0 and 0.1, such as 0.05.

Alternatively or in addition, the capsule body 110 includes a plurality of pores 118 thereon. The pores 118 may have a diameter on the nano-scale, for example less than a micron in length. The pores 118 are each sized to modify the kinetics of the transfer of therapeutic agent 120 contained within the hollow interior space 116 of the capsule 100 to exit the hollow interior space 116 into spaces outside of the capsule's 100 volume. Specially, the capsule body 110 is configured to absorb and desorb the therapeutic agent 120. In use of the capsule, the therapeutic agent 120 is dissolved in into the capsule body 110 inner surface 114, and slowly desorbed from the capsule body 110 outer surface 112 to move to the other side of the capsule body 110. The pores 180 are included in the capsule body 110 to tune the release kinetics of the therapeutic agent 120. Without the pores, the kinetics can be much slower. The pores 118 have a diameter dₚ and the therapeutic agent 120 has a diameter dₘ. The pores 118 are configured to tune the release kinetics of the therapeutic agent 120 from the hollow interior space 116. The capsule body 110 further includes an inner radius Rᵢ extending from an central axis 130, extending axially through the capsule body 110 from a first axial end 132 to a second axial end 134, to the inner surface 114 and an outer radius Rₒ extending from the central axis 130 to the outer surface 112. The difference in the lengths of the outer radius Rₒ and the inner radius Rᵢ is a measurement of a thickness of the capsule body 110. Furthermore, a length of the capsule body 110 may be measured by the distance from the first axial end 132 to the second axial end 134.

The therapeutic agent 120 may be any agent that provides therapeutic relief. For example, the therapeutic agent 120 may be an antibody, which may be configured to combat viral pathogens, an anti-inflammatory agent, which may be configured to provide relief to inflammatory diseases such as tumor necrosis factor alpha-induced inflammation, or another suitable drug or therapeutic agent. In some examples, therapeutic agent 120 is an anti-cancer antibody such as bevacizumab, ranibizumab, gemtuzaumab, alemtuzumab, rituximab, trastuzumab, nimotuzumab, cetuximab, panitumumab, bavituximab, or combinations thereof. Alternatively or in addition, the therapeutic agent 120 may include anti-cancer drugs such as, but not limited to, doxorubicin, methotrexate, and combinations thereof. Alternatively or in addition, the therapeutic agent 120 may include an anti-inflammatory antibody such as infliximab, adalimumab, ustekinumab, basiliximab, daclizumab, omalizumab, or combinations thereof. Alternatively or in addition, the therapeutic agent 120 may include anti-microbial drugs such as, but not limited to, vancomycin, levofloxacin, or combinations thereof. Alternatively or in addition, the therapeutic agent 120 may include TGF beta inhibitors such as, but not limited to, pirfenidone. Alternatively or in addition, the therapeutic agent 120 may include anti-antibodies. For example, the therapeutic agent 120 may include anti-VEGFs such sunitnib, sorafenib, axitinib, pazopanib, or combinations thereof. Alternatively or in addition, the therapeutic agent 120 may be an anti-TGF beta such as, but not limited to, Fresolimumab. Alternatively or in addition, the therapeutic agent 120 may be an anti-SARS-CoV2 such as, but not limited to, Sotrovimab, bamlanivimab/estesvimab, carsirivimab/imdeviab, or combinations thereof. Alternatively or in addition, the therapeutic agent 120 may be another antibody such as palivizumab, abciximab, or combinations thereof. Alternatively or in addition, the therapeutic agent 120 may include genes in the form of mRNA and siRNA. Indeed, any of the previously listed therapeutic agents 120 may be included as the therapeutic agent 120 in the nano-porous capsule 100, in any combination. In some examples, more than one species of therapeutic agents 120 may be included in the hollow interior space 116, each of these species of therapeutic agents 120 may be therapeutic in treating the same or different ailment suffered by the recipient of the nano-porous capsule implant 100.

As will be explained below, the thickness of the capsule body 110, the polymer included in the capsule body 110, the length of the capsule body 110, the pore 118 diameter dₚ, and the therapeutic agent 120 diameter dₘ, each may be manipulated to adjust the release rate of the therapeutic agent 120 from the hollow interior space 116 of the capsule body 110, and accordingly, the therapeutic agent 120 may be configured to be released from the capsule implant 100 over a predetermined amount of time after having been administered to a recipient such as a patient. In this way, predetermined sizes of pores 118 and therapeutic agents 120, and choices of therapeutic agents 120 and polymers included in the capsule body 110 may be selected as desired for release rate.

Referring to FIG. 2, methods 200 of making the nano-porous capsule implant 100 are also provided. In an embodiment of the method 200 of making the nano-porous capsule implant 100, a plurality of sheets 202 of a polymer are stacked 210 onto each other to form a stack 204 of sheets 202 of polymer. The stack 204 of sheets of polymer may then be rolled 220 circumferentially to form a generally cylindrical shape 206 to define a generally cylindrical space configured to receive the therapeutic agent 120 to be deposited therein. In some embodiments, the polymer included in the polymer sheets 202 are selected from a group consisting of polycaprolactone (PCL), poly(lactic acid) (PLA), 90:10 poly(lactic-co-glycolide) (PLGA), and 50:50 PLGA, and combinations thereof. The generally cylindrical shape 206 defines a hollow interior cylindrical space 214 having a first open end 20 a at one end of the generally cylindrical interior space 214, and a second open end 208b at another end of the generally cylindrical interior space 214 opposite the first end 208a. The generally cylindrical shape 206 may then be submerged (not shown) in deionized water resulting in pores 180 being formed through the generally cylindrical shape 206. Alternatively, or in addition, the generally cylindrical shape 206 may be subjected to sonication resulting in pores 180 being formed through the generally cylindrical shape 206. Regardless of the method to form the pores 180 in the generally cylindrical shape 206, the hollow interior cylindrical space 214 is then filled with a desired therapeutic agent 120 via the first open end 208a or the second open end 208b of the generally cylindrical shape 206. Each of the first end 208a and the second end 208b are then sealed 250, forming the nano-porous capsule implant 100 defining the hollow interior space 116. As a result of sealing the ends 208a, 208b of the generally cylindrical shape 206, the first end 132 and the second end 134 of the capsule body 110 and formed. In this way, the therapeutic agent 120 is housed within the hollow interior space 116, though the therapeutic agent 120 may still exit the hollow interior space 116 via the pores 118. After formation of the nano-porous capsule implant 100, the implant 100 may be inserted into a syringe 260 and injected or otherwise delivered to a recipient of the nano-porous capsule implant 100. The number of polymer sheets 202 stacked 210 prior to rolling 220 the stacked polymer sheets to form the generally cylindrical shape 206 determines the thickness of the eventually formed capsule body 110.

With continued reference to FIG. 2, optionally, the generally cylindrical shape 206 may be cut 230 transversely, either before or after forming pores 118 through the generally cylindrical shape 206. Cutting the generally cylindrical shape 206 in this way forms two or more generally cylindrical shapes 206a, 206b from the original generally cylindrical shape 206. Similarly, each of these newly formed generally cylindrical shapes 206a, 206b includes open ends; for example generally cylindrical shape 206a may include open end 208a from the original generally cylindrical shape 206 as well as a newly formed open end 208c. Similarly, generally cylindrical shape 206b may include open end 208b from the original generally cylindrical shape 206 as well as a newly formed open end 208d. The cutting 230 step may be repeated as desired to form a desired amount of generally cylindrical shapes 206 to be filled 240 with therapeutic agent 120.

The polymer sheets may be synthesized prior to their stacking onto each other to form the nano-porous capsule implant 100. For example, the nano-porous polymer sheets may be synthesized by first creating a polymer solution including the polymer(s) selected to eventually form the capsule body 110 in DCM. This solution may furthermore include a desired, predetermined amount of polyethylene glycol (PEG) as a porogen for differing porosities of the capsule body 110. For example, polyethylene glycol may be added to the solution at a ratio between 0.0 and 0.1 PEG to polymer by weight, for example 0.5 PEG to polymer by weight. The solution, thus mixed, may then be placed in a mold floating in a bath sonicator to promote curing of the polymer sheets. The mold may be covered with parafilm and sonicated for a duration of between 40 minutes and 120 minutes, or as otherwise needed, such as for 80 minutes, to create a homogeneous distribution of porogen. The DCM may then be evaporated from the polymer sheets. After sonication, the polymer sheet may be removed, and air dried in a chemical hood at room temperature overnight. A plurality of polymer sheets, thus formed, may be stacked, rolled, and sealed to form the capsule body 110, as described above.

EXAMPLES

### Materials

Poly(caprolactone) (MW 65,000-75,000) was purchased from PolyScitech®, Inc. (West Lafayette, IN). Dichloromethane (DCM), sodium hydroxide (NaOH), and polyethylene glycol (PEG, Average MW 3350) were purchased from Fisher Chemical® (USA). Bevacizumab (Bev) was purchased from Pfizer® (Zirabev®) (New York, NY).

### PCL Sheet Synthesis

The nano-porous PCL sheet was synthesized by first creating a 50 mg/mL PCL solution in DCM with varying amounts of polyethylene glycol (PEG) as a porogen for differing porosities. The ratios were 0.0, 0.05 and 0.1 PEG to PCL by weight. 800 μL of the PCL solution was placed in a mold floating in a bath sonicator at 15° C. The mold was covered with parafilm and sonicated at 50% power for 80 minutes to create a homogeneous distribution of porogen and slowly evaporate the DCM. After sonication, the sheet was removed, and air dried in a chemical hood at room temperature overnight.

### Example 1: Implant Fabrication

To create PCL implants, the PCL sheet was cut into 0.5×1 cm² pieces using a razor blade. The pieces were then rolled around a 22-gauge needle at 45° C. to create a double layered tube 1 cm in length and 0.0946 cm in diameter. The tube was then submerged in deionized water overnight to leach out the PEG and create a porous structure and then dried overnight at room temperature. Once dried, a 60° C. iron was used to clamp one end of the tube shut and a 26-gauge needle was used to load 4 μL of a Bev solution into the tube. Once the Bev solution was loaded, the iron was used to seal the other end shut and then both ends were superglued to prevent any leakage that may occur. The nano-porous capsule implant fabrication process is shown in FIG. 2.

### Example 2: Drug Release Profile

Implants loaded with the Bev solution were placed in 1 mL of PBS and incubated at 35° C. to mimic the temperature of the human eye. The PBS solution was removed and replaced with fresh PBS every 24 h, 72 h, or 7 days depending on how long the implant has been incubated. The removed solution was analyzed using UV-Vis at 280 nm to determine the Bev concentration. Using the concentration, the amount of Bev released was plotted to determine the release profile.

### Example 3: Accelerated Drug Release Profile

The implants used in these experiments were prepared in the same way as Example 2. However, instead of placing the implants in 1 mL of polybutylene succinate (PBS), the implants were placed in 1 mL of 0.1 M NaOH at 35° C. for accelerated release. Every 24 h the NaOH solution was removed, analyzed using UV-Vis at 285 nm due to the shift in Bev in NaOH, and fresh NaOH was added. The study was conducted over 14 days due to the breakdown of 0.1 PEG PCL implants. The concentration found was used to determine the amount of Bev released each day and then plotted.

### Example 4: SEM Imaging

The surface and cross-section structures were analyzed using scanning electron microscopy (SEM) (Thermo Fisher Scios Dual Beam SEM®, Hillsboro, OR). The PCL sheet or tube was placed on a SEM sample holder with dual sided graphite conductive tape. The samples were sputter-coated with 5 nm platinum/gold nanoparticles for 10 s using Denton Vacuum Desk II® (Moorestown, NJ). To obtain the cross-section view, tubes were submerged in glycerol, placed in liquid nitrogen until frozen, and then fractured to get the best cross-section view without having the edges roll from cutting at room temperature. The samples were then rinsed with DI water and dried overnight. The mean pore sizes and pore density were analyzed using ImageJ® (NIH).

The surface and cross-section structures were analyzed using scanning electron microscopy (SEM) (Thermo Fisher Scios Dual Beam SEM®, Hillsboro, OR). The PCL sheet or tube was placed on a SEM sample holder with dual sided graphite conductive tape. The samples were sputter-coated with 5 nm platinum/gold nanoparticles for 10 s using Denton Vacuum Desk II® (Moorestown, NJ). To obtain the cross-section view, tubes were submerged in glycerol, placed in liquid nitrogen until frozen, and then fractured to get the best cross-section view without having the edges roll from cutting at room temperature. The samples were then rinsed with DI water and dried overnight. The mean pore sizes and pore density were analyzed using ImageJ® (NIH).

Porosity was calculated using the following equation:

$$ε = \frac{{V}_{b} - {V}_{t}}{{V}_{b}} \qquad ( 1 )$$

Where V_{b} is the bulk volume (volume of membrane if no pores) and Vₜ is the true volume of the membrane (volume of membrane taking out pore volume). The bulk volume was calculated using the thickness of the membrane determined by SEM and the area of the polymer sheet used to roll the implants. The true volume is the bulk volume minus the total volume of the pores in the membrane. The volume of pores was determined by finding the volume of a pore using the average pore diameter (determined by SEM) and assuming spherical pores, the pore density (pores/nm³ determined by SEM), and the total volume of the implant. Multiplying the pore density and volume gives the total number of pores, which multiplied by the average pore volume gives the total volume of pores. This method may underestimate the porosity because of the SEM image resolution.

### Example 5: Permeability

The permeability experiments were conducted using a vertical permeability column. The donor side was filled with 200 μL of 50 mg/mL Bev solution and the receiver side was filled with 5 mL of PBS. The membrane used was a 1 cm×1cm double layered PCL sheet. The membrane was made by placing two 1×1 cm PCL sheets on top of each other and pressing them together at 45° C. to create a double layered membrane. The membranes were soaked in DI water for 24 h to remove the PEG and then placed in the column without drying. Once the membrane was securely placed between the donor and receiver sections, 200 μL of the donor solution was loaded and then the opening was sealed off to reduce evaporation altering the concentration of the donor and receiver solutions. 500 μL of the receiver and 10 μL of the donor solution were removed every 72 h to check the concentration using UV-Vis. The removed solutions were replaced by fresh PBS for the receiver solution and the donor was replaced by fresh donor solution or completely replaced depending on the remaining concentration.

After 12 days, the mass of Bev that permeated the membrane was plotted against time to get dQ/dt in the equation:

$$\begin{matrix} & {C}_{i} \cdot P = \frac{1}{A} \cdot \frac{d Q}{d t} \\ {C}_{i} \cdot P = \frac{1}{A} \cdot \frac{d Q}{d t} & \end{matrix} \qquad ( 2 )$$

where Cᵢ is the initial concentration (donor solution concentration), P is the permeability coefficient, and A is the area of the exposed membrane.

### Example 6: Partition Coefficient

The partition coefficient K is required for the release kinetics model equation. To measure the partition coefficient, a 0.5×0.5 cm² double layered PCL membrane was made in a similar way to the membranes in the permeability experiment. The membranes were soaked in 200 μL of PBS for 48 h. After 48 h the membranes were blotted with a Kimwipe to remove surface liquid and then weighed to determine the wet mass of the membrane. The membranes were then dried overnight in a chemical hood. The dry membranes were then placed in 200 μL of an equilibrium Bev solution for 72 h to allow the membranes to absorb the Bev from the solution. After 72 h the membranes were removed from the equilibrium, blotted, and placed in 200 μL of fresh PBS to extract Bev for 48 h. This extraction process was repeated until the amount extracted was 10% of the first extraction. The remaining equilibrium solution and all extraction solutions were analyzed using UV-Vis to determine the Bev concentration. The partition coefficient was then determined using the equation below.

$$K = \frac{\frac{M e}{W}}{\frac{{M}_{e q}}{V * ρ}} \qquad ( 3 )$$

where Mₑ is the total mass extracted, W is the wet mass of the membrane, M_{eq} is the amount of Bev left in the equilibrium solution, V is the volume of water left in the equilibrium solution, and ρ is the density of the solution.

### The Modeling Equations

The equation used to model the release profile is

$$\frac{{M}_{t}}{{M}_{∞}} = 1 - exp [ - \frac{( {R}_{i} L + {R}_{o} L + 2 {R}_{i} {R}_{o} ) {D}_{e} Kt}{{R}_{i}^{2} L ( {R}_{o} - {R}_{i} )} ] \qquad ( 4 )$$

where Mₜ is the mass released at time t, M_{∞}, is the mass loaded in the implant, Rᵢ is the inner radius of the implant, Rₒ is outer radius of the implant, Dₑ is the effective diffusivity, K is the partition coefficient, and L is the length of the implant. This equation models Fick's law of diffusion of molecules from the lumen of a cylindrical implant through a membrane wall. Rₒ, Rᵢ, and L can all be measured from the implants, but Dₑ and K must be determined experimentally or calculated from other parameters that can be determined experimentally to create this model. K may be determined experimentally, as described herein. To find Dₑ, permeability, Stokes-Einstein diffusion, hindrance factor for diffusion, hindrance factor, and the restrictive factor all were determined experimentally or calculated.

The permeability of Bev across the PCL membrane can be described by

$$P = \frac{ε H D}{τ h} \qquad ( 5 )$$

where ε is the porosity of the membrane, H is the overall hinderance factor for diffusion, τ is tortuosity, h is the thickness of the membrane, and D is the Stokes-Einstein diffusion coefficient.

$$D = \frac{{k}_{B} T}{6 π η r} \qquad ( 6 )$$

where k_{B} is the Boltzmann constant, T is the temperature in Kelvin, η is the viscosity of water at 25° C., and r is the hydrodynamic radius of Bev. The overall hindrance factor for diffusion, H, is expressed as:

$$H ( λ ) = \frac{6 {π ( 1 - λ )}^{2}}{{K}_{t}} \qquad ( 7 )$$

where λ is the ratio of permeant radius to pore radius and Kₜ, the hinderance factor, is calculated by:

$${K}_{t} = \frac{9}{4} {{π}^{2 \sqrt{2}} ( 1 - λ )}^{- \frac{5}{2}} [ 1 + {\sum }_{n = 1}^{2} {{a}_{n} ( 1 - λ )}^{n} ] + {\sum }_{n = 0}^{4} {a}_{n + 3} {λ}^{η} \qquad ( 8 )$$

The last value that needs to be determined for Equation 3 is effective diffusivity (Dₑ). Dₑ can be determined from the equation:

$${D}_{e} = \frac{ε D}{τ} \cdot {K}_{r} \qquad ( 9 )$$

where Kᵣ is the restrictive factor when the ratio of the molecule diameter (dₘ) and pore diameter (dₚ) is less than 1. The relationship between the diameters is expressed by:

$${K}_{r} = {[ 1 - \frac{{d}_{m}}{{d}_{p}} ]}^{4} \qquad ( 10 )$$

Solving Equation 5 for tortuosity and substituting it into Equation 9 provides an equation for De that does not require tortuosity to calculate.

$${D}_{e} = \frac{{PhK}_{r}}{H} \qquad ( 11 )$$

### Characterization of the PCL Capsule

PCL sheets with 3 PEG ratios (0.1, 0.05, and 0.0) were synthesized. The overall structure of the implant was imaged using scanning electron microscopy. The scale-like structure on the surface is most likely due to the evaporation of DCM during the sheet preparation. It was confirmed that the outer surface 112 exhibited a scale like structure and the inner surface 114 was smooth except for small indents created by the presence of PEG. The pore size, porosity, and thickness of the PCL implants with 3 PEG ratios were analyzed using the cross-section SEM images. 0.0 PEG PCL sheets exhibited no pores on the surfaces 112, 114 or in the cross section and the average thickness is 20.5 μm±0.248 μm, the 0.05 PEG ratio had a mean pore size of 521.5 nm±26.1 nm, a porosity of 3.14±0.354%, an average scale area of 5.48E3±993.5 μm², and an average thickness of 25.6 μm±0.180 μm, and the 0.1 PEG ratio has an average pore size of 621 nm±24 nm, a porosity of 6.88E−2±6.72E−3, and an average thickness of 27.6 μm±0.541 μm. Based on the data, increasing the PEG concentration increases the pore size (p=0.0068), increases porosity (p=4.8E−4), and increases the thickness of the sheet (p=3.52E−4 for No PEG and 0.05 PEG, p=1.04E−8 for No PEG and 0.1 PEG, and p=3.48E−4 for 0.05 PEG and 0.1 PEG). Results of this example are shown in Table 1A.

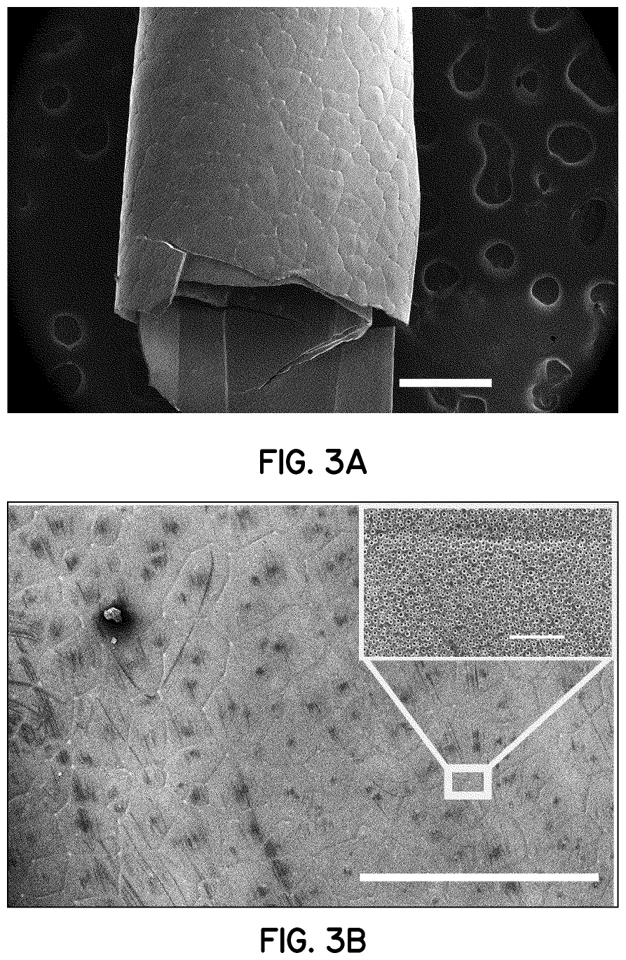

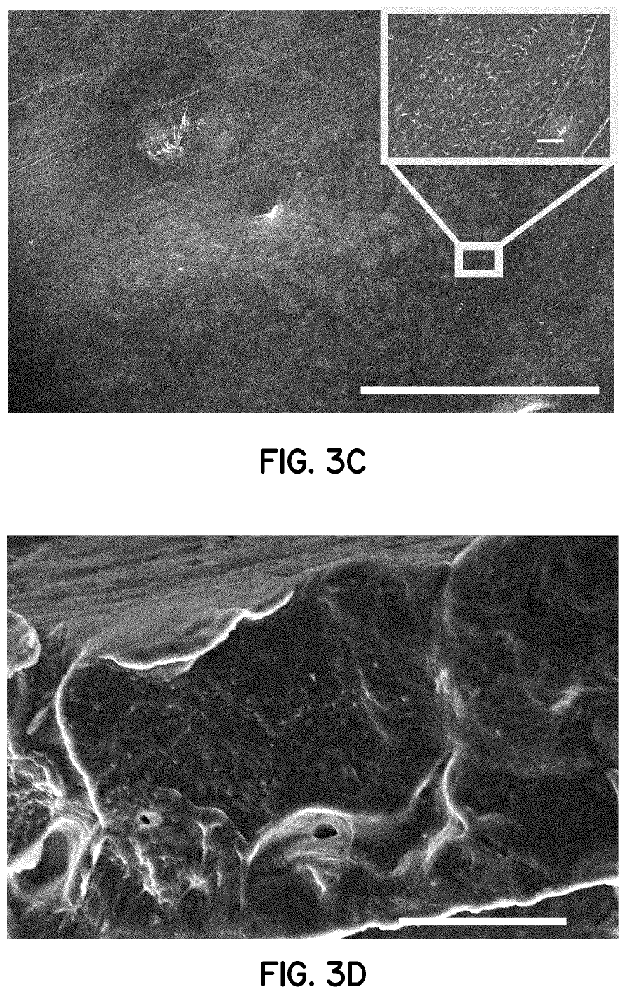

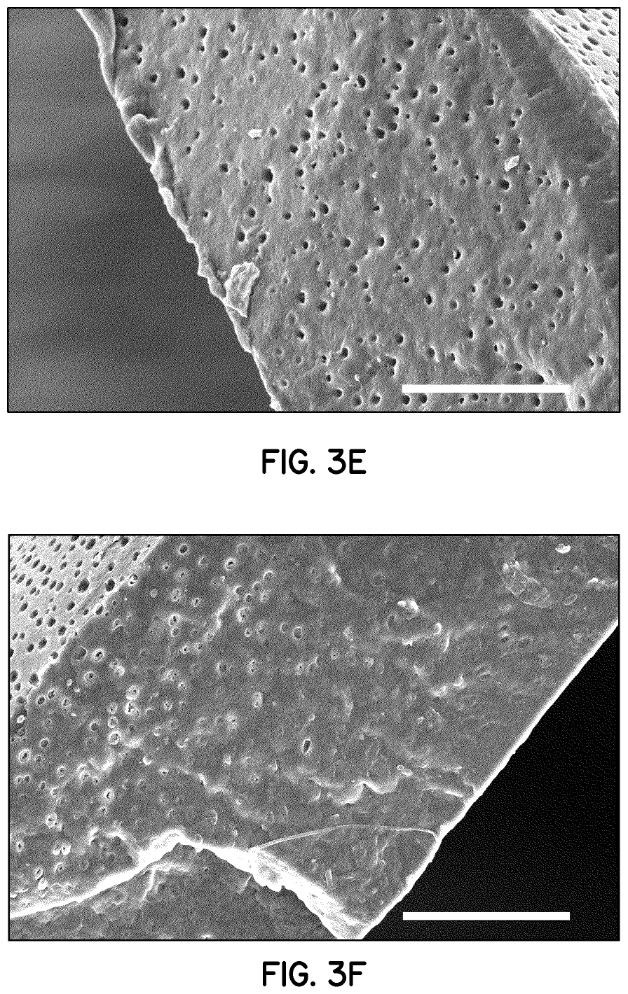

A second example following the preparatory procedures stated above was conducted. PCL sheets with three PEG ratios (0.0, 0.05, and 0.1) were synthesized. The overall structure of the implant was imaged using SEM. Particularly, FIG. 3A an SEM image of a 0.05 PEG PCL overall (scale bar 500 μm) according to an embodiment of the invention. FIG. 3B an SEM image of a 0.05 PEG PCL sheet top according to an embodiment of the invention. Scale bars=10 μm. FIG. 3C an SEM image of a 0.05 PEG PCL sheet bottom according to an embodiment of the invention. Scale bars=10 μm. FIG. 3D is a cross-section of a 0.0 PEG PCL according to an embodiment of the invention. Scale bars=10 μm. FIG. 3E is a cross-section of a 0.05 PEG PCL according to an embodiment of the invention. Scale bars=10 μm. FIG. 3F is a cross-section of a 0.05 PEG PCL according to an embodiment of the invention. Scale bars=10 μm.

The scalelike structure on the surface is most likely due to the evaporation of DCM during the sheet preparation. It was confirmed that the outer surface 112 exhibited a scalelike structure and the inner surface 114 was smooth except for small indents created by the presence of PEG. The area of the scale structure (μm²) was significantly different for all three PEG conditions. 0.0, 0.5, and 0.1 PEG had an average area of 200±10, 5480±500, and 3130±220 μm², respectively, indicating that PEG affects the surface morphology (p-value<0.05). Along with the scale structure, the outer surface 112 of the sheet has a high density of pores homogeneously distributed across the entire surface 112 (inset of FIG. 3B). The pore size, porosity, and thickness of the PCL implants with three PEG ratios were analyzed using the cross-sectional SEM images (FIGS. 3D-F and Table 1B). 0.0 PEG PCL sheets exhibited no pores on the surface or in the cross section. The 0.05 PEG ratio had a mean pore size of 520±20 nm and a porosity of 3.14%. The 0.1 PEG ratio had an average pore size of 620±10 nm and a porosity of 6.88%. The pore size (p=0.0068) and the porosity (p=4.8E−4) statistically significantly increased from 0.05 to 0.1 PEG ratio. The average thickness of 0.0, 0.05, and 0.1 PEG was 20.5±0.25, 27.6±0.54, and 25.6±0.18 μm, respectively, showing no correlation between the thickness and the PEG ratio.

### Drug (Bev) Release Profile from the PCL Implants

The release profile from a 1.25 mg implant shows that 87% of the Bev has been released after 6 months. The overall release is quick in the first 51 days releasing 67.1%±5.8% before slowing down to release 27.3%±6.67% in the next 185 days. The daily amount released also varies as time increases going from an average of 32.8±5.95 μg/day in the first week to an average of 1.15±0.20 μg/day in the last month.

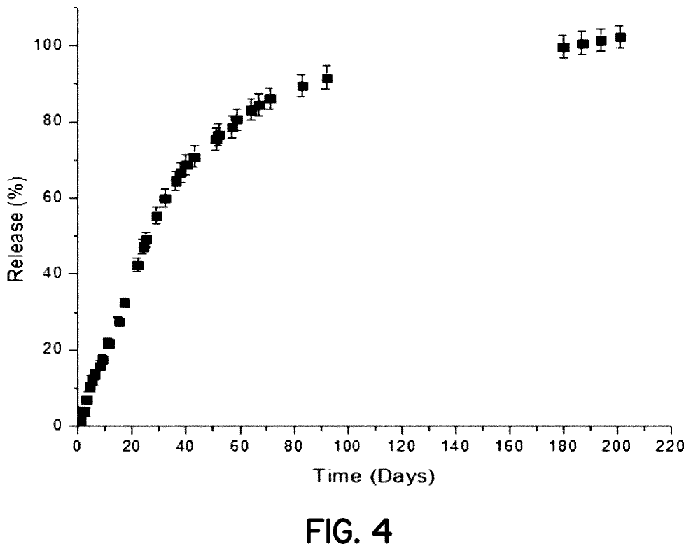

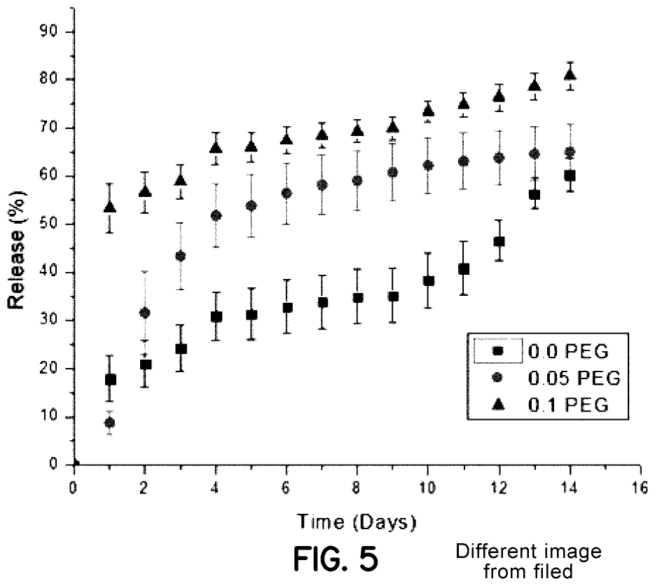

Continuing from the description of the second example above, the release profile from the implant shows that 97.7% of the Bev was released after 6 months (FIG. 4). The release was fast in the first —50 days, releasing 75% before slowing down. release 23% in the next 160 days. The decrease in the release rate with time is due to the drop in the concentration gradient across the membrane as Bev diffuses out of the implant. The release curve shown in FIG. 4 exhibits first-order kinetics.

### Drug Release Profile Under Accelerated Degradation Conditions

Because hydrolysis is the method of degradation for PCL, utilizing NaOH allows for an accelerated look at the release curve for varying PEG to PCL conditions. Utilizing the accelerated conditions allows for a sample release curve to be created without having to wait for 6 months. 0.1 M NaOH was chosen based on the speed of the implant degradation. Higher concentrations of NaOH broke down the implants too quickly and lower concentrations did not break down quickly enough. The curves from the 0.05 PEG PCL accelerated release mimic the first order shape from the natural condition release. Using the similar curve shapes, a time correlation can be obtained to create a relationship between time in 0.1 M NaOH and time in natural condition.

It took more than 30, 20-30, and 10-20 days to observe the breakage of the polymer capsule for 0.0, 0.05, and 0.1 PEG, respectively, in 0.1 M NaOH. In 0.5 M NaOH, it took 20-30, 2-3, and 1-2 days to observe the breakage for 0.0, 0.05, and 0.1 PEG, which is too fast to analyze the surface characterization. In 0.05 M NaOH, it took longer than 30 days for all conditions, which is too long for an accelerated test. These results also suggest that the amount of OH⁻ necessary for accelerated degradation depends on the polymer capsule morphology, including the porosity. The higher the porosity, the faster degradation because the pores increase surface areas for OH⁻ ions to penetrate and interact with the polymer. The curves from the 0.05 PEG PCL accelerated release mimic the first-order kinetics from the natural condition release. 0.1 PEG has a 53.4±6.78% burst release in the first 24 h before slowing down. The burst release from 0.1 PEG PLGA could be due to the high porosity, allowing for more Bev to be released in a shorter time compared to 0.0 and 0.05 PEG. The 0.0 PEG ratio has a 2-step release curve with a small amount (31.0±7.37%) released in the first 5 days before plateauing until day 10 where the release speeds back up. This second surge of release is due to microcracks forming in the implant due to degradation, which might have occurred earlier for 0.05 and 0.1 PEG. Using the curve shapes, a time correlation can be obtained to create a relationship between the time in 0.1 M NaOH and the time in natural conditions.

TABLE 1A
Pore size, porosity, and thickness analysis from
Natural Degradation SEM cross-section images

| Month | | 0.05 PEG |
|---|---|---|
| 1 | Pore Diameter (nm) | 374.5 ± 22.1 |
| | Porosity (%) | 0.671 ± 0.039 |
| | Scale Area (μm²) | 256.8 ± 46.0 |
| | Sheet thickness (μm) | 19.6 ± 0.23 |
| 2 | Pore Diameter (nm) | 377.8 ± 17.1 |
| | Porosity (%) | 0.412 ± 0.040 |
| | Scale Area (μm²) | 4.13E3 ± 968.5 |
| | Sheet thickness (μm) | 24.2 ± 0.36 |
| 3 | Pore Diameter (nm) | 424.4 ± 22.2 |
| | Porosity (%) | 0.353 ± 0.018 |
| | Scale Area (μm²) | 3.84E3 ± 684.1 |
| | Sheet thickness (μm) | 22.3 ± 1.00 |
| 4 | Pore Diameter (nm) | 341.1 ± 20.4 |
| | Porosity (%) | 0.600 ± 0.044 |
| | Scale Area (μm²) | 8.81E3 ± 811.0 |
| | Sheet thickness (μm) | 27.7 ± 0.38 |
| 5 | Pore Diameter (nm) | 430.9 ± 22.9 |
| | Porosity (%) | 0.454 ± 0.028 |
| | Scale Area (μm²) | 13.87E3 ± 2.01E3 |
| | Sheet thickness (μm) | 29.0 ± 1.22 |
| 6 | Pore Diameter (nm) | 526.7 ± 31.0 |
| | Porosity (%) | 0.337 ± 0.055 |
| | Scale Area (μm²) | 17.79E3 ± 2.54E3 |
| | Sheet thickness (μm) | 27.3 ± 0.85 |

TABLE 1B
Pore size, Porosity, Scale Area, and Thickness Analysis of PCL Capsules

| Month | | 0.0 PEG | 0.05 PEG | 0.1 PEG |
|---|---|---|---|---|
| 0 | Pore Diameter (nm) | N/A | 510 ± 30 | 620 ± 20 |
| | Porosity (%) | N/A | 3.14 ± 0.177 | 6.88 ± 0.336 |
| | Scale Area (μm²) | 200 ± 10 | 5480 ± 990 | 3130 ± 220 |
| | Sheet thickness (μm) | 20.5 ± 0.25 | 25.6 ± 0.18 | 27.6 ± 0.54 |

Because hydrolysis is the method of degradation for PCL, utilizing NaOH allows for an accelerated look at the release curve for varying PEG to PCL conditions taking 2 weeks to achieve a sample release curve instead of 6 months. 0.1 M NaOH was chosen because it degraded the implants quickly, but slow enough to allow for in depth analysis of the degradation. Higher concentrations of NaOH (0.25 and 0.5 M) broke down the implants too quickly and a lower concentration at 0.05 M did not break down quickly enough (data not shown). The curves from the 0.05 PEG PCL accelerated release mimic the first-order shape from the natural condition release. 0.1 PEG has a 53.4%±6.78% burst release in the first 24 hours before slowing down to also mimic first-order kinetics. The 0.0 PEG ratio has a 2-step release curve with a small amount (31.0%±7.37%) released in the first 5 days before plateauing until day 10 where the release speeds back up. This second surge of release is due to micro cracks forming in the implant due to degradation. Using the curve shapes, a time correlation can be obtained to create a relationship between time in 0.1 M NaOH and time in natural condition.

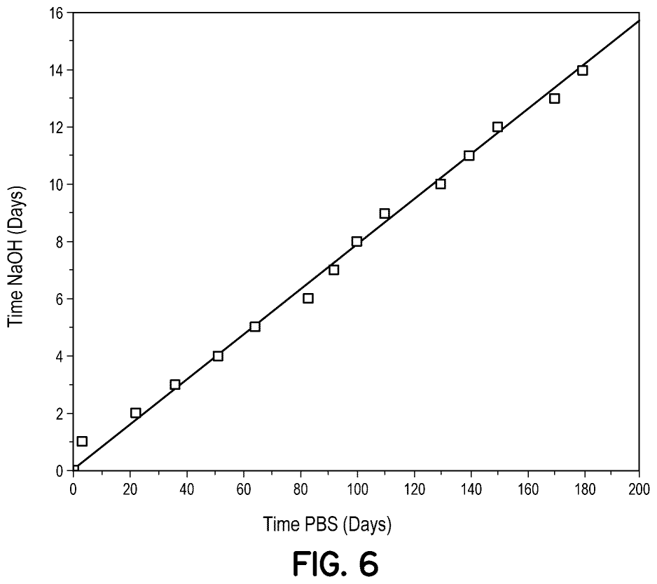

FIG. 6 shows a linear relationship between time in NaOH and natural condition. The equation obtained from the graph explains the mathematical relation between time in the two conditions. By setting the release percent at 14 days as equivalent to the release at 180 days, the other points on the accelerated curve can be compared to free release points based on area under the curve. Using this equation allows for future accelerated condition experiments to be translated to natural conditions without the need to spend 6 months per experiment. Similar results were achieved for the second example, shown in FIG. 6. Particularly, FIG. 6 shows a correlation of the time frame for the drug release between the accelerated and natural conditions. The equation obtained from the graph explains the mathematical relation between the time in the two conditions. By multiplying the actual release by a factor of 0.663 (the ratio of the accelerated final release and actual final release), the time in PBS and NaOH can be plotted to find the mathematical relationship between the time in each solution. Using this equation allows for future accelerated condition experiments to be translated to natural conditions without the need to spend 6 months per experiment. The time relation is described:

t_{A}=0.0807*t_{N} (12)

In this equation, t_{A} is the time in NaOH solution and t_{N} is the time in PBS. This specific correlation is specific to the 0.05 PEG PCL capsule.

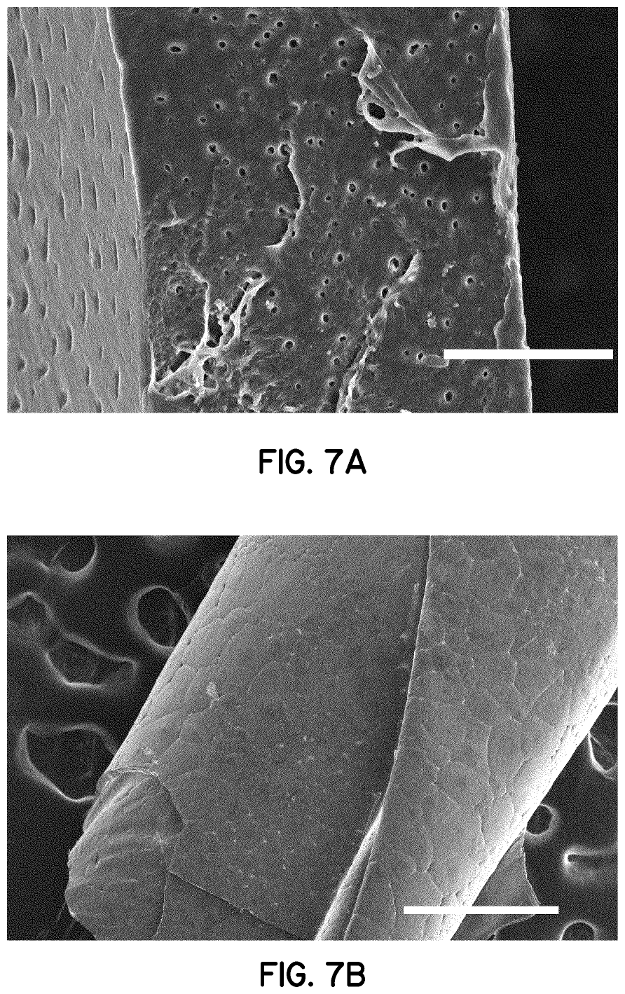

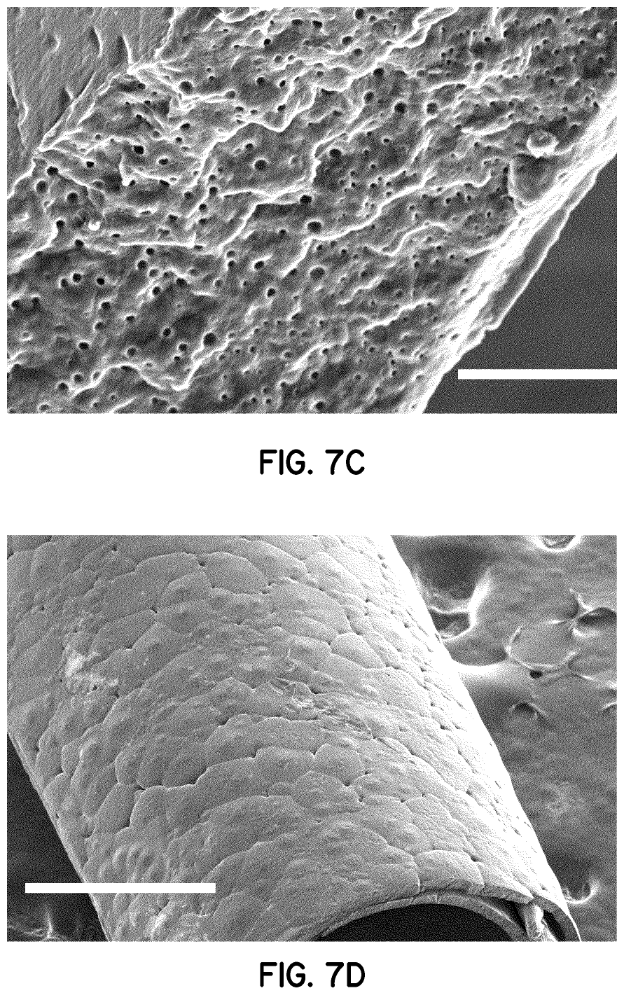

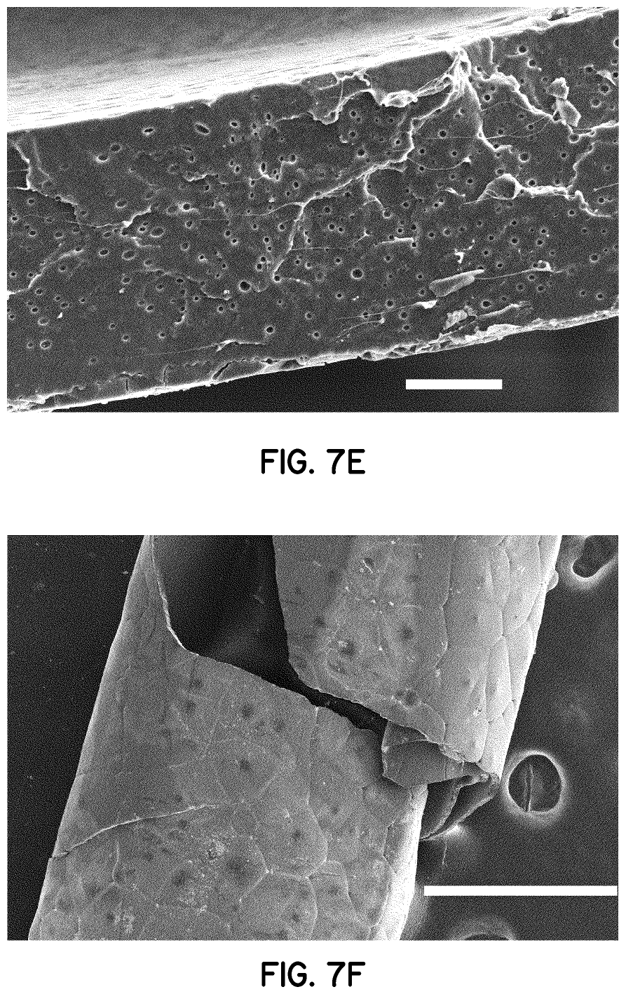

Based on the images in FIGS. 7A-7F and SEM images at 2, 4, 6 months, the main effects of degradation are shown in the changes in the scale structure over time. Particularly, FIG. 7A is a cross-sectional SEM image of a 0.05 PEG capsule after 2 months of natural degradation. FIG. 7B is an overall SEM image of a 0.05 PEG capsule after 2 months of natural degradation. FIG. 7C is a cross-sectional SEM image of a 0.05 PEG capsule after 4 months of natural degradation. FIG. 7D is an overall SEM image of a 0.05 PEG capsule after 4 months of natural degradation. FIG. 7E is a cross-sectional SEM image of a 0.05 PEG capsule after 6 months of natural degradation. FIG. 7F is an overall SEM image of a 0.05 PEG capsule after 6 months of natural degradation. Scale bars=10 μm for cross-section and 500 μm for overall images. As degradation occurs, the scale area drops in the first month from 5.48E3±993.5 μμm² to 256.8±46.0 μm² and then begins to increase as degradation continues (p<0.05) except between month 2 and 3 where there is no significant difference (p>0.05). The pore size shrinks from day 0 to 1 month of degradation decreasing from 512.5±26.1 nm to 374.5±22.1 nm (p=2.35E−5) and then stays consistent until month 6 where it increases to a size similar to day 0 (p=0.462). The porosity stays the same for the first month (p=0.871), decreases after 2 months (p=3.37E−4), stays consistent through month 4 then increases at month 5 (p=2.125E−3) and stays consistent through month 6 (p=0.165).

SEM imaging allows determination of pore size, porosity, and the effects of degradation over time. Using NaOH to speed up degradation allows observation of the effects of degradation over one month. Based on the time correlation established earlier, one month in NaOH is equivalent to 383 days in normal condition. After analyzing the images, the effects of degradation begin to take effect after 4 days in NaOH. The no PEG condition develops grooves in the cross section, the 0.05 PEG condition begins to develop smaller, more frequent pores, and 0.1 PEG increases pore size and small cracks can be observed on the exterior surface. After 10 days, the No PEG develops micron sized cracks frequently across the exterior surface, the 0.05 PEG develops small cracks on the surface, the pore size begins to increase, porosity increases, and the implant becomes brittle. The 0.1 PEG condition develops large cracks on the surface, pore size and porosity increase, and the implant begins to breakdown. After 20 days, the No PEG condition's cracks are no longer just on the surface but begin to go through the membrane and holes begin to form at the intersections of scales. The 0.05 PEG condition develops very large cracks that penetrate the membrane, pore size and porosity increase, a wave like texture forms on the surface, the outer layer begins to flake off and the implant is extremely brittle. The 0.1 PEG condition has completely broken down and is only comprised of chunks from the implant. After 30 days, the no PEG condition's cracks and holes increase in size and the surface changes to a rough wave like texture compared to the smooth surface seen earlier. The 0.05 PEG has completely broken down and only chunks of the implant remain. The chunks of the 0.1 PEG have continued to degrade, and now only small flakes remain.

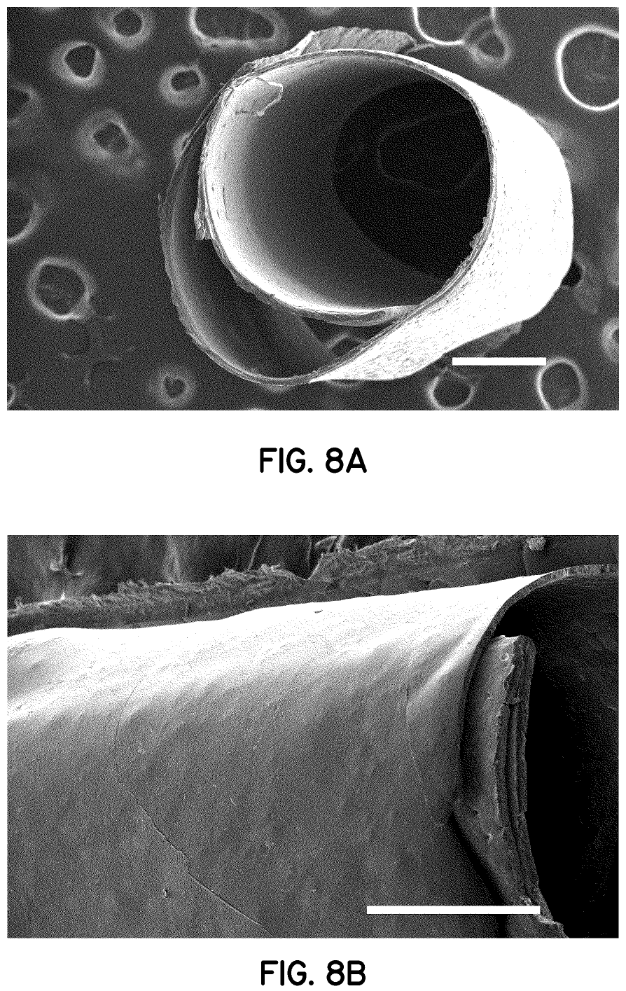

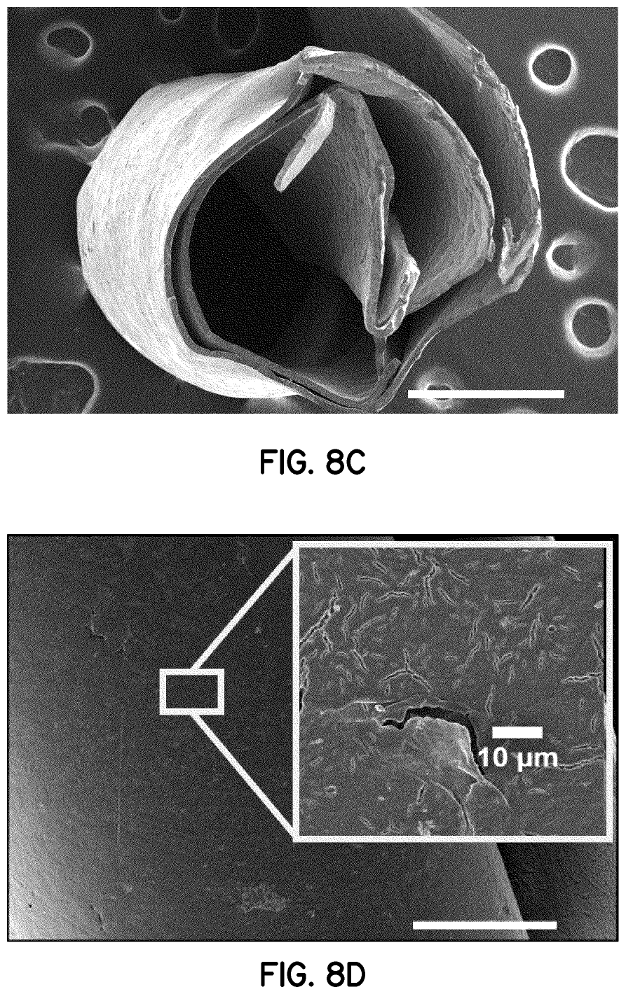

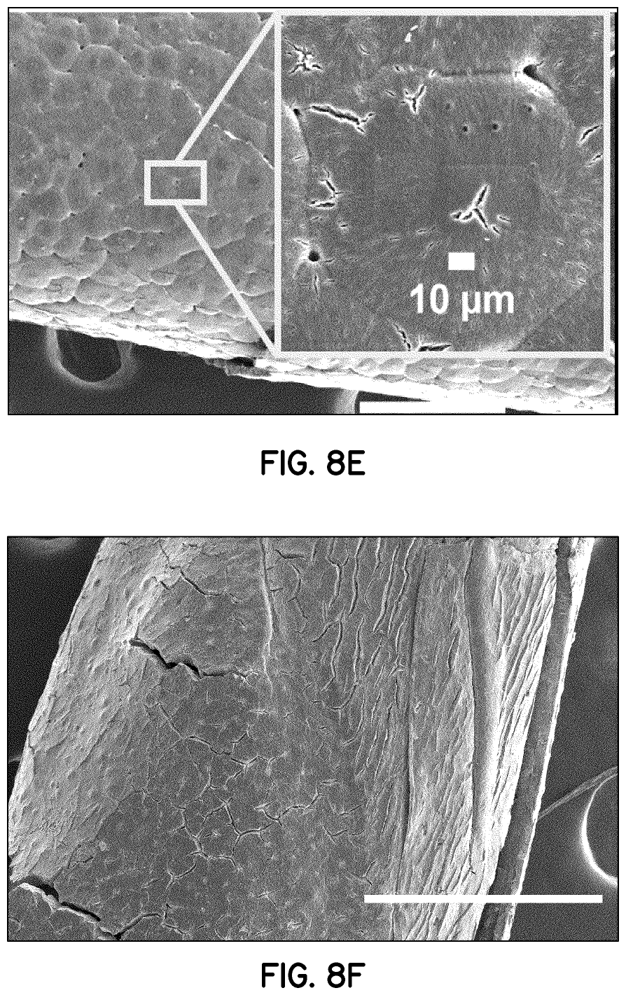

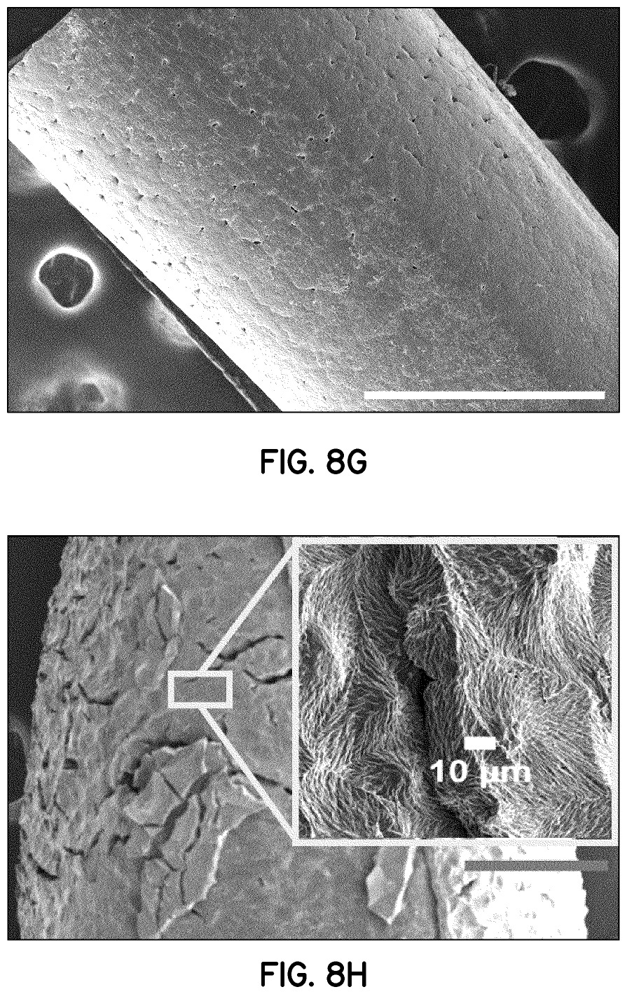

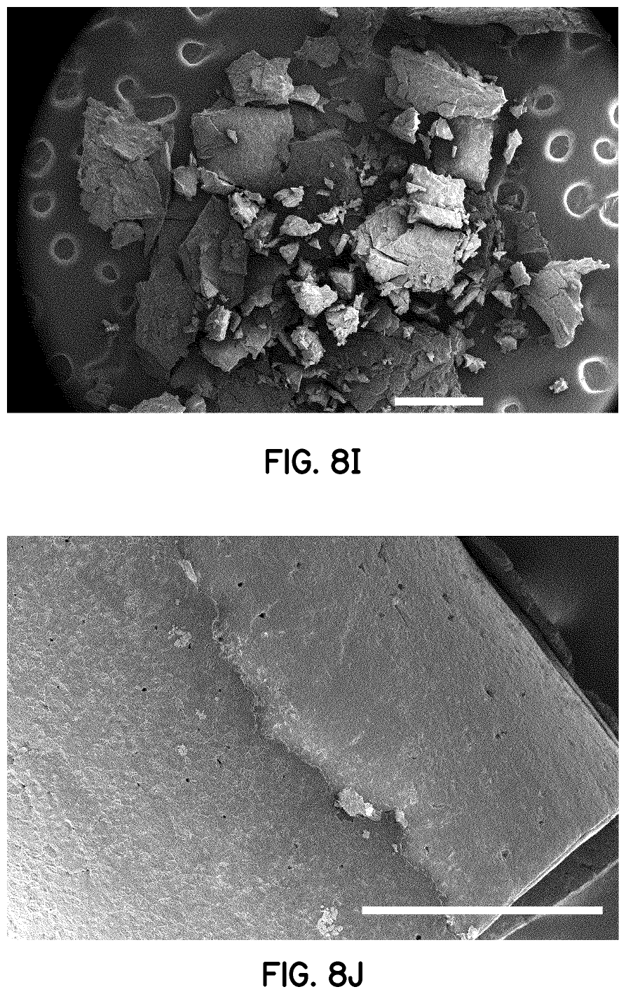

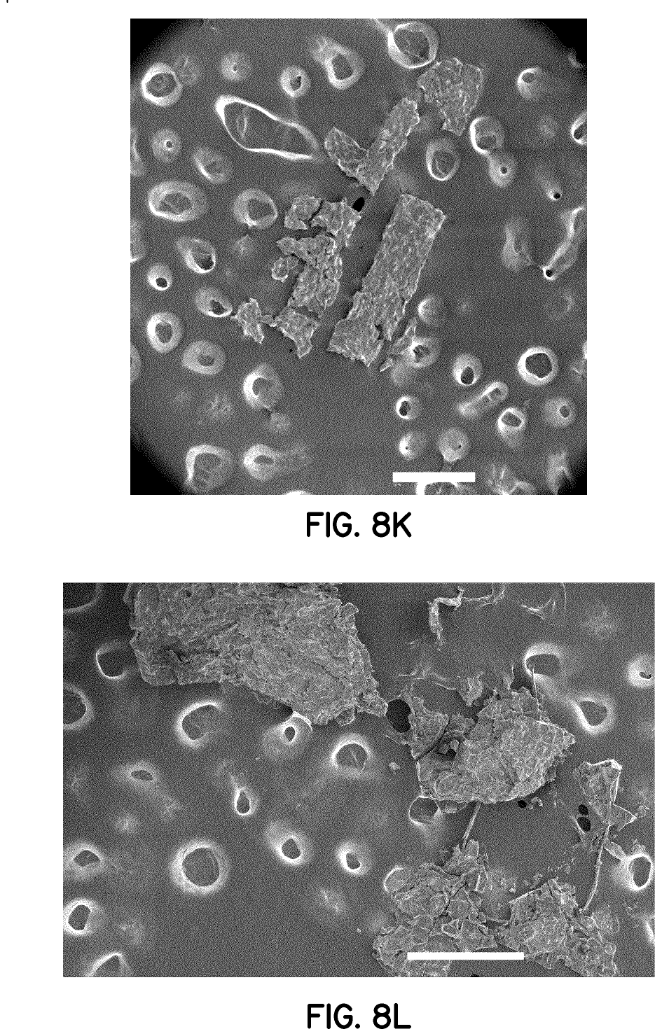

The no-PEG-ratio does not have any pores, nor do a significant amount form during the degradation process so there are no measurements for porosity and pore size in Table 1. Measurements for 0.05 PEG 30 days and 0.1 PEG 20 and 30 days have no measurements in Table 1 due to these conditions being degraded to the point of debris, as seen in FIGS. 8A-J. Despite the cracks forming, the no PEG condition showed no significant changes until 30 days of degradation. After 30 days, the thickness significantly decreased by close to 50% (p=2.73E−4).

The 0.05 PEG pore diameter dropped in size at day 4 from 512.5±26.1 nm to 390.7±16.5 nm (p=1.17E−5), exhibited no change by day 10, and then grew back to the original size at day 20 increasing from 389.8 nm±16.4 nm to 626.5 nm±22.8 nm (p=0.220 between day 0 and 20). The porosity did not change until day 10 where it decreased from 3.19±0.195% to 2.22±0.215% (p=0.011) and stayed steady through day 20. Similar to the natural degradation, the scale area drastically decreases from 5.48E3±993.5 μm² to 760.0±90.1 μm². After 4 days, the scale area increases to 7.69E3±744.4 μm² before decreasing to 4.43E3±315.3 μm² at day 20. The thickness decreased in the first 4 days of degradation (p=3.32E−5), held steady though 10 days, and then continued to decrease through day 20 (p=0.0174).

0.1 PEG had no significant change in pore size throughout the degradation process, the only changes were in porosity, which steadily increased, and thickness, which initially decreased and then held steady until the implant became debris. The porosity of the 0.1 PEG continuously increased as degradation continued (p<0.05) and the thickness decreased in the first 4 days (p=0.00129) but did not change significantly between day 4 and day 10 (p=0.211).

TABLE 2
Pore size, porosity, and thickness analysis from
Accelerated Degradation SEM cross-section images

| | | No PEG | 0.05 PEG | 0.1 PEG |
|---|---|---|---|---|
| Day | | Accelerated | Accelerated | Accelerated |
| 0 | Pore Diameter (nm) | N/A | 512.5 ± 26.1 | 621 ± 24 |
| | Porosity (%) | N/A | 3.14 ± 0.354 | 6.88 ± 0.672 |
| | Scale Area (μm²) | N/A | 5.48E3 ± 993.5 | N/A |
| | Sheet thickness (μm) | 20.5 ± 0.25 | 25.6 ± 0.18 | 27.6 ± 0.54 |
| 4 | Pore Diameter (nm) | N/A | 390.7 ± 16.5 | 596.3 ± 26.7 |
| | Porosity (%) | N/A | 3.19 ± 0.195 | 9.04 ± 0.242 |
| | Scale Area (μm²) | N/A | 760.0 ± 90.1 | N/A |
| | Sheet thickness (μm) | 20.6 ± 1.51 | 18.7 ± 0.21 | 23.8 ± 0.71 |
| 10 | Pore Diameter (nm) | N/A | 389.8 ± 16.4 | 694.6 ± 38.1 |
| | Porosity (%) | N/A | 2.22 ± 0.215 | 5.28 ± 0.534 |
| | Scale Area (μm²) | N/A | 7.69E3 ± 744.4 | N/A |
| | Sheet thickness (μm) | 20.2 ± 0.81 | 20.7 ± 0.88 | 30.3 ± 1.23 |
| 20 | Pore Diameter (nm) | N/A | 626.5 ± 22.8 | N/A |
| | Porosity (%) | N/A | 2.87 ± 0.173 | N/A |
| | Scale Area (μm²) | N/A | 4.43E3 ± 315.3 | N/A |
| | Sheet thickness (μm) | 20.9 ± 0.40 | 17.0 ± 0.58 | N/A |
| 30 | Pore Diameter (nm) | N/A | N/A | N/A |
| | Porosity (%) | N/A | N/A | N/A |
| | Scale Area (μm²) | N/A | N/A | N/A |
| | Sheet thickness (μm) | 11.5 ± 0.28 | N/A | N/A |

Both degradation conditions showed a similar trend in pore size with the diameters decreasing after the initial degradation and then increasing at the end to reach the initial pore size before degradation. The porosity trends began the same with the initial parts of degradation showing no effect on porosity and then decreasing, but the natural degradation saw an increase at 5 months while the accelerated degradation did not increase before it completely degraded. Due to the different degradation mechanisms for natural and accelerated, the thickness trends were completely different. The more consistent degradation in the natural condition caused no trend to appear in the thickness of the membranes while the increased surface degradation in the accelerated condition caused the thickness to decrease as degradation continued. The scale area allowed verification of the time correlation developed. The day 4 scale area is between the 1 month and 2-month scale areas (p=4.58E−5 and 1.31E−3 respectively) which lines up with the time analysis (55 days) and the 10 day scale area was not significantly different from the 4-month scale area (p=0.337) which corresponds to the time analysis of 127 days for 10 days of accelerated degradation.

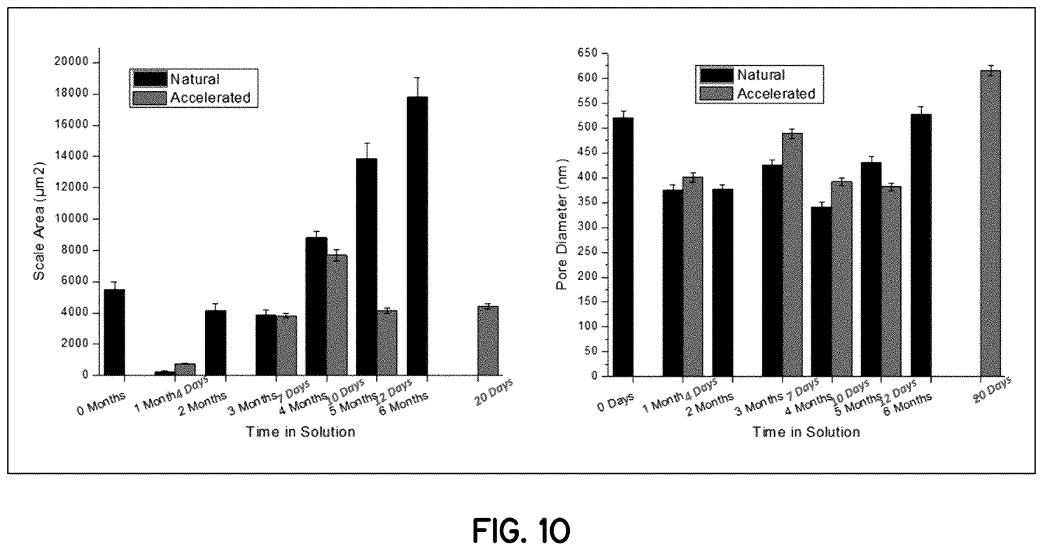

Regarding the second experiment, based on the SEM images at 2, 4, and 6 months, as represented in FIGS. 7A-7F, the main effects of degradation are the changes in the scale structure over time. As degradation occurred, the scale area dropped in the first month from 5480±990 to 260±50 m2 (Table 2) and then began to increase as degradation continues (p<0.05) except between months 2 and 3 where there is no significant difference (p>0.05). The pore size decreased from day 0 to month 1 of degradation from 510±30 to 370±20 nm (Table 3; p=2.35E−5) and then stayed consistent until month 6 where it increased. The porosity stayed the same for the first month, decreased after 2 months, stayed consistent through month 4, then increased at month 5, and stayed consistent through month 6. The trend for 0.05 PEG is represented as a bar graph in FIG. 10, combined with the accelerated degradation results.

Using a 0.1 M NaOH solution, accelerated degradation was achieved. The 0.05 PEG pore diameter decreased from 510±30 to 390±20 nm (p=1.17E−5) at day 4 of the accelerated condition, exhibited no change by day 10, and then increased at day 20, increasing from 390±20 to 630±20 nm (Table 4). The porosity did not change until day 10 where it decreased from 3.19±0.097 to 2.22±0.108% (p=0.011) and stayed steady through day 20 (Table 4) Similar to the natural degradation, the scale area drastically decreases from 5480±990 to 760±90 m². After 4 days, the scale area increases to 7690±740 m² before decreasing to 4430±320 m² at day 20. The thickness decreased in the first 4 days of degradation (p=3.32E−5), held steady through 10 days, and then continued to decrease through day 20 (p=0.0174).

The 0.0 PEG ratio does not have any pores; thus, there are no measurements for porosity and pore size in Table 4. Measurements for 0.05 PEG 30 days and 0.1 PEG 20 and 30 days have no measurements in Table 4 because a significant breakdown of the structure with debris was observed, as seen in FIGS. 8A-8J. Specifically, FIG. 8A is an overall SEM image of a 0.0 PEG capsule after 4 days of accelerated degradation. FIG. 8B is an overall SEM image of a 0.0 PEG capsule after 10 days of accelerated degradation. FIG. 8C is an overall SEM image of a 0.0 PEG capsule after 20 days of accelerated degradation. FIG. 8D is an overall SEM image of a 0.0 PEG capsule after 30 days of accelerated degradation. FIG. 8E is an overall SEM image of a 0.05 PEG capsule after 4 days of accelerated degradation. FIG. 8F is an overall SEM image of a 0.05 PEG capsule after 10 days of accelerated degradation. FIG. 8G is an overall SEM image of a 0.05 PEG capsule after 20 days of accelerated degradation. FIG. 8H is an overall SEM image of a 0.05 PEG capsule after 30 days of accelerated degradation. FIG. 8I is an overall SEM image of a 0.1 PEG capsule after 4 days of accelerated degradation. FIG. 8J is an overall SEM image of a 0.1 PEG capsule after 10 days of accelerated degradation. FIG. 8K is an overall SEM image of a 0.1 PEG capsule after 20 days of accelerated degradation. FIG. 8L is an overall SEM image of a 0.1 PEG capsule after 30 days of accelerated degradation. Scale bars=500 μm for all images.

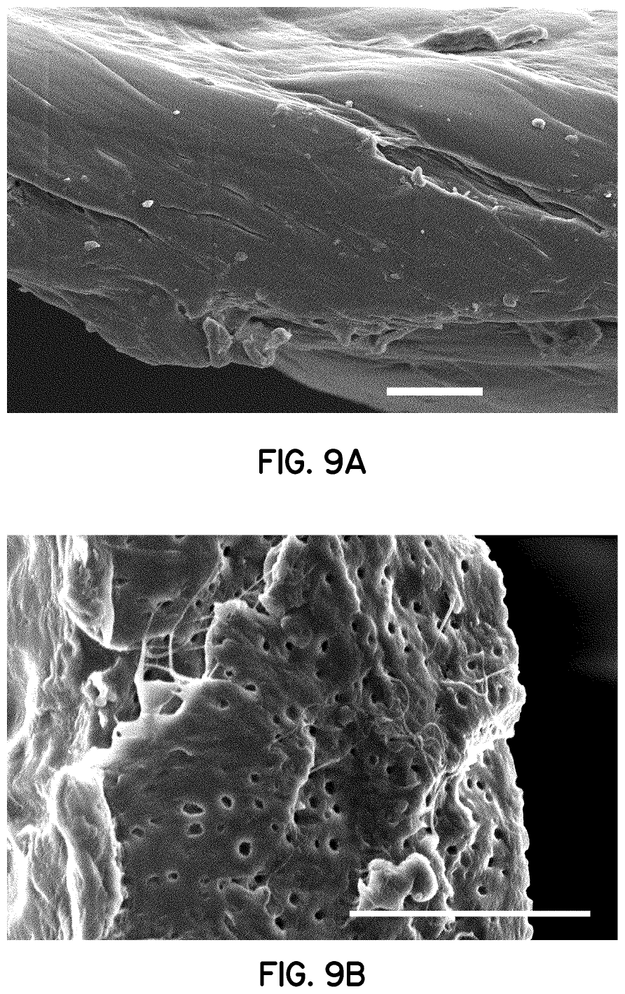

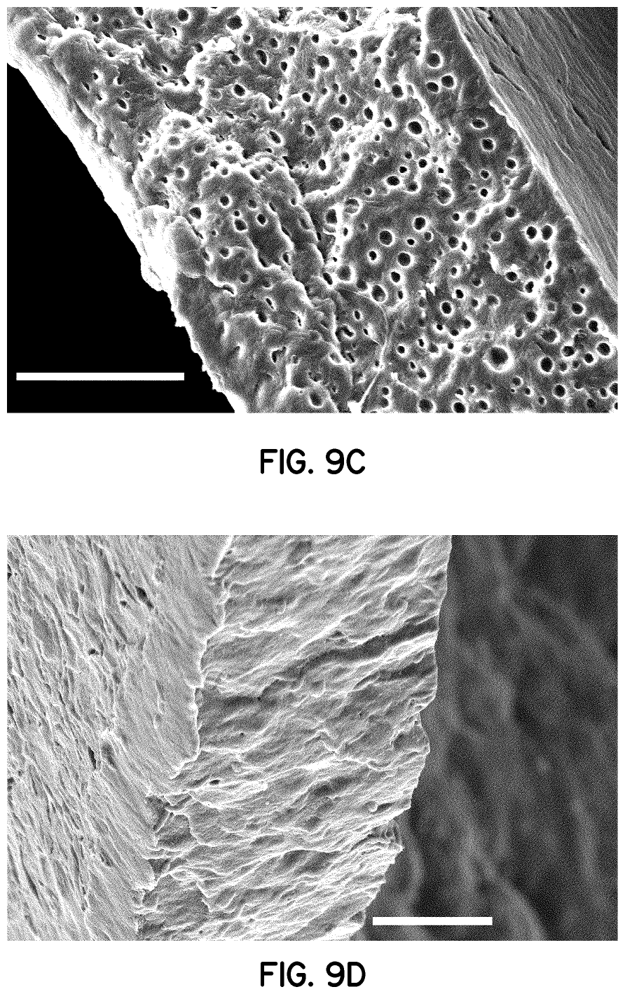

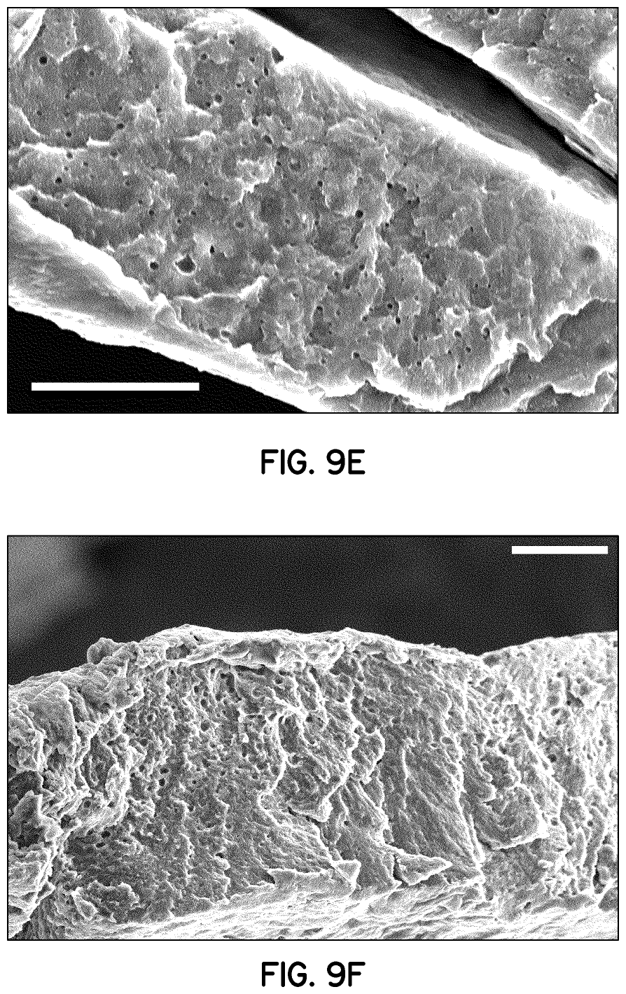

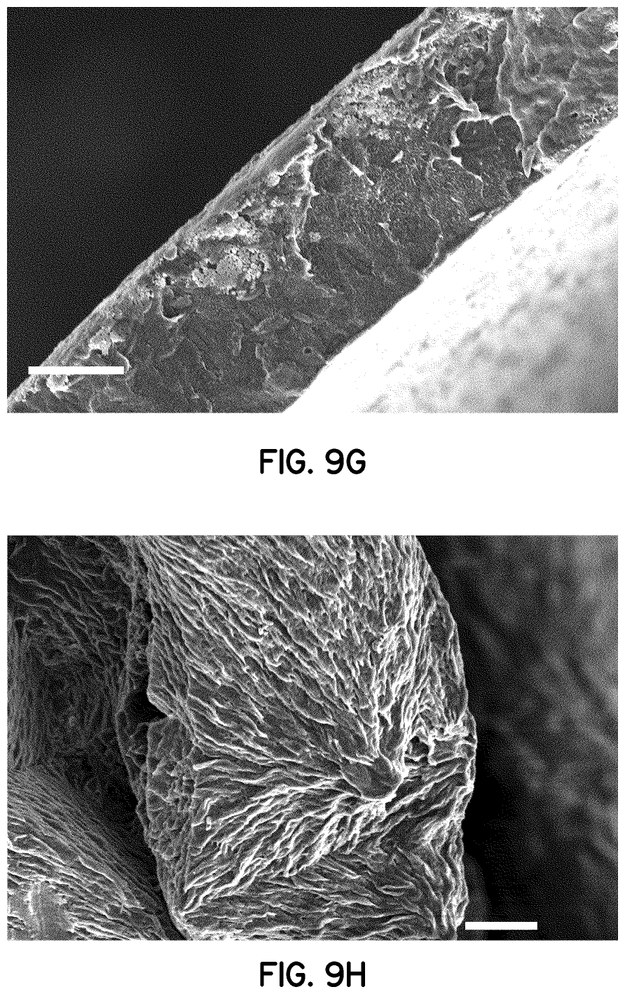

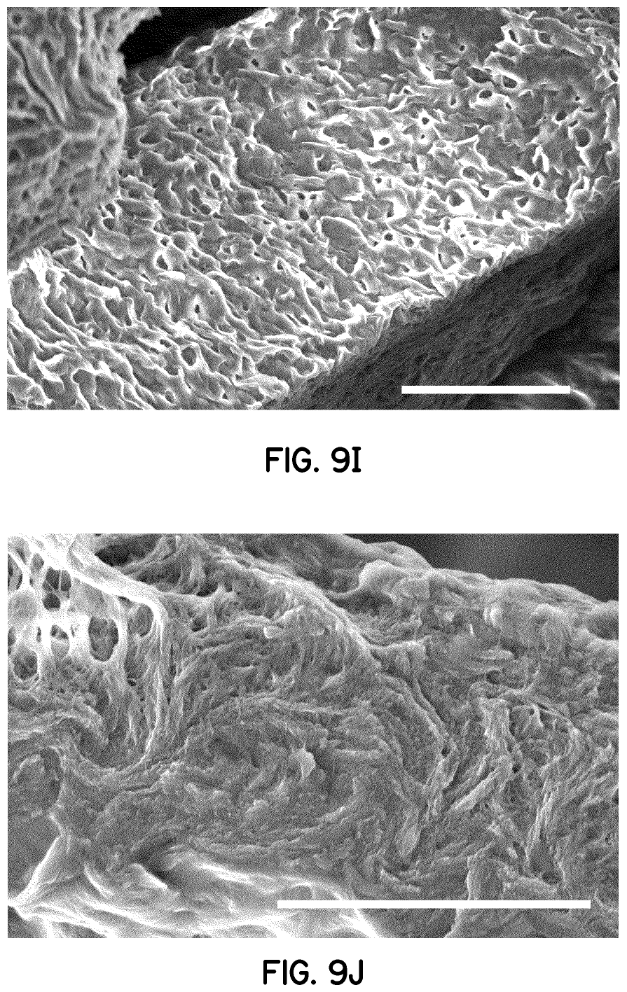

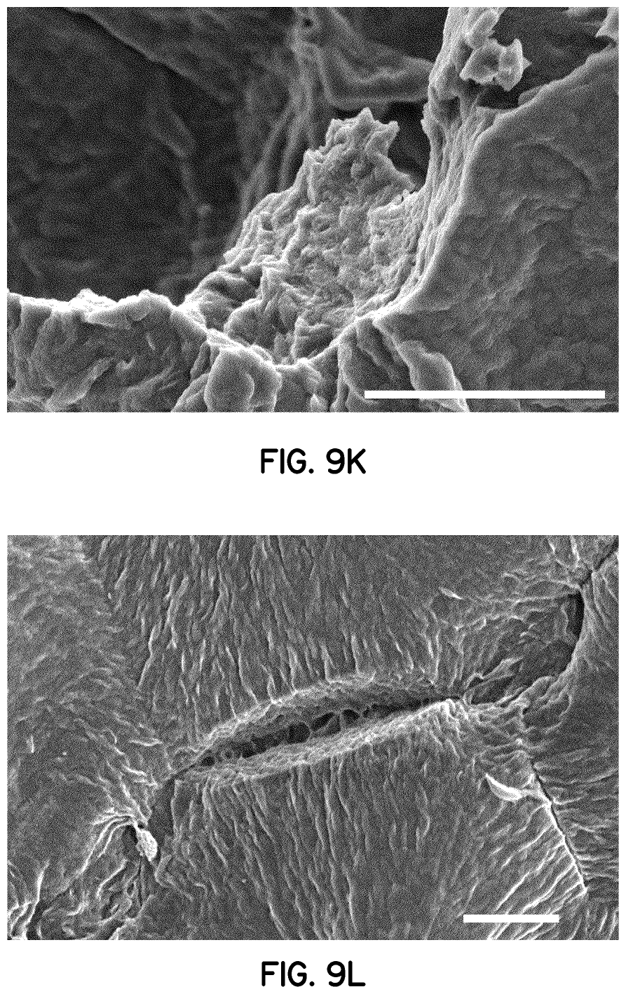

Despite the cracks forming, the 0.0 PEG condition showed no significant changes until 30 days of degradation (FIG. 8G). After 30 days, the thickness significantly decreased by close to 50% (p=2.73E−4). 0.1 PEG had no significant change in pore size throughout the degradation process, and the only changes were in porosity and thickness. The porosity increased in the first 4 days of degradation from 6.88±0.336 to 9.03±0.121% (p=0.011) and then decreased to 5.28±0.267% (p=0.0211). The thickness decreased in the first 4 days (p=0.00129) but did not change significantly between days 4 and 10 (p=0.211). The trend of scale area and pore diameter is represented in FIG. 10, integrated with the natural degradation considering the time correlation between the natural and accelerated conditions (FIGS. 9A-J). Specifically, FIG. 9A is a cross-sectional SEM image of a 0.0 PEG capsule after 4 days of accelerated degradation. FIG. 9B is a cross-sectional SEM image of a 0.0 PEG capsule after 10 days of accelerated degradation. FIG. 9C is a cross-sectional SEM image of a 0.0 PEG capsule after 20 days of accelerated degradation. FIG. 9D is a cross-sectional SEM image of a 0.0 PEG capsule after 30 days of accelerated degradation. FIG. 9E is a cross-sectional SEM image of a 0.05 PEG capsule after 4 days of accelerated degradation. FIG. 9F is a cross-sectional SEM image of a 0.05 PEG capsule after 10 days of accelerated degradation. FIG. 9G is a cross-sectional SEM image of a 0.05 PEG capsule after 20 days of accelerated degradation. FIG. 9H is a cross-sectional SEM image of a 0.05 PEG capsule after 30 days of accelerated degradation. FIG. 9I is a cross-sectional SEM image of a 0.1 PEG capsule after 4 days of accelerated degradation. FIG. 9J is a cross-sectional SEM image of a 0.1 PEG capsule after 10 days of accelerated degradation. FIG. 9K is a cross-sectional SEM image of a 0.1 PEG capsule after 20 days of accelerated degradation. FIG. 9L is a cross-sectional SEM image of a 0.1 PEG capsule after 30 days of accelerated degradation. Scale bars=10 μm for all images.

Both degradation conditions showed a similar trend in pore size and scale area, decreasing after the initial degradation and then increasing at the end (FIG. 10). Utilizing Equation 12, the corresponding time points in PBS for 4, 7, 10, 12, and 20 days in NaOH are 50, 90, 124, 130, and 248 days, respectively. To help verify these time correlations, statistical analysis of the scale area and pore diameter between accelerated and natural conditions was used as comparisons. The scale area at day 4 in the accelerated degradation condition (red columns) was between the 1 and 2 month scale areas in the natural degradation condition. The scale area of 4 day is larger than 1 month (p=4.58E−5) and smaller than 2 months (p=0.0013), matching the time correlation, 50 days. The pore diameter for 1 months, 2 months, and 4 days shows no significant difference (p>0.05 for all). This puts 4 days somewhere between 1 and 2 months, meaning that 50 days in PBS is an accurate representation of 4 days in NaOH. A 7 day pore diameter (488.7±10.0 nm) was not significantly different from the 3 months pore diameter (p>0.05), and the scale area (3827.2±96.3 m²) was also not significantly different from the 3 month scale area (p=0.05). The scale area at day 10 was not significantly different from the month 4 scale area (p=0.337) and neither was the pore diameter (p=0.045). The similarity in scale area and slight difference in pore diameter help to verify 124 days in PBS as an accurate representation for 10 days in NaOH. Twelve days began to see some deviation in the scale area. The pore diameter (393.6±7.4 nm) was not significantly different from 5 months (p>0.05), but the 12 day scale area (4150.3±183.6 m²) was significantly smaller than 5 months in PBS (p=0.016). The deviation of the scale area during the end of the degradation conditions is most likely due to the increased surface degradation in the NaOH conditions compared to that of the PBS. The porosity trend was also the same between natural and accelerated degradation conditions, showing a decrease in porosity and an increase later. However, the trend of thickness showed the difference between natural and accelerated degradation. The consistent degradation in the natural condition caused no trend to appear in the thickness of the membranes, while the increased surface degradation in the accelerated condition caused the thickness to decrease as degradation continued, implying that surface erosion is dominant in the accelerated condition.

TABLE 3
Pore size, porosity, and thickness analysis from
Natural Degradation SEM cross-section images

| Month | | Natural Degradation |
|---|---|---|
| | Pore Diameter (nm) | 370 ± 20 |
| | Porosity (%) | 3.07 ± 0.089 |
| | Scale Area (μm²) | 260 ± 50 |
| | Sheet thickness (μm) | 19.6 ± 0.23 |
| 2 | Pore Diameter (nm) | 380 ± 20 |
| | Porosity (%) | 1.94 ± 0.095 |
| | Scale Area (μm²) | 4130 ± 970 |
| | Sheet thickness (μm) | 24.2 ± 0.36 |
| 3 | Pore Diameter (nm) | 420 ± 20 |
| | Porosity (%) | 2.36 ± 0.059 |
| | Scale Area (μm²) | 3840 ± 680 |
| | Sheet thickness (μm) | 22.3 ± 1.00 |
| 4 | Pore Diameter (nm) | 340 ± 20 |
| | Porosity (%) | 2.08 ± 0.076 |
| | Scale Area (μm²) | 8810 ± 810 |
| | Sheet thickness (μm) | 27.7 ± 0.38 |
| 5 | Pore Diameter (nm) | 430 ± 20 |
| | Porosity (%) | 3.17 ± 0.096 |
| | Scale Area (μm²) | 13870 ± 2010 |
| | Sheet thickness (μm) | 39.0 ± 1.22 |
| 6 | Pore Diameter (nm) | 530 ± 30 |
| | Porosity (%) | 4.29 ± 0.351 |
| | Scale Area (μm²) | 17790 ± 2540 |
| | Sheet thickness (μm) | 27.3 ± 0.85 |

TABLE 4
Pore size, porosity, and thickness analysis from
Accelerated Degradation SEM cross-section images

| | | No PEG | 0.05 PEG | 0.1 PEG |
|---|---|---|---|---|
| Day | | Accelerated | Accelerated | Accelerated |
| 4 | Pore Diameter (nm) | N/A | 390.7 ± 16.5 | 596.3 ± 26.7 |
| | Porosity (%) | N/A | 3.19 ± 0.195 | 9.04 ± 0.242 |
| | Scale Area (μm²) | 2384 ± 100 | 390 ± 20 | 690 ± 0.242 |
| | Sheet thickness (μm) | 20.6 ± 1.51 | 18.7 ± 0.21 | 23.8 ± 0.71 |
| 10 | Pore Diameter (nm) | N/A | 389.8 ± 16.4 | 694.6 ± 38.1 |
| | Porosity (%) | N/A | 20.2 ± 0.81 | 5.28 ± 0.534 |
| | Scale Area (μm²) | 150 ± 10 | 389.8 ± 16.4 | 4360 ± 300 |
| | Sheet thickness (μm) | 20.2 ± 0.81 | 20.7 ± 0.88 | 30.3 ± 1.23 |
| 20 | Pore Diameter (nm) | N/A | 626.5 ± 22.8 | N/A |
| | Porosity (%) | N/A | 2.87 ± 0.173 | N/A |
| | Scale Area (μm²) | 1720 ± 70 | 4.43E3 ± 320 | N/A |
| | Sheet thickness (μm) | 20.9 ± 0.40 | 17.0 ± 0.58 | N/A |
| 30 | Pore Diameter (nm) | N/A | N/A | N/A |
| | Porosity (%) | N/A | N/A | N/A |
| | Scale Area (μm²) | 180 ± 10 | N/A | N/A |
| | Sheet thickness (μm) | 11.5 ± 0.28 | N/A | N/A |

### Effective Diffusion Coefficient (De) and Partition Coefficient (k)

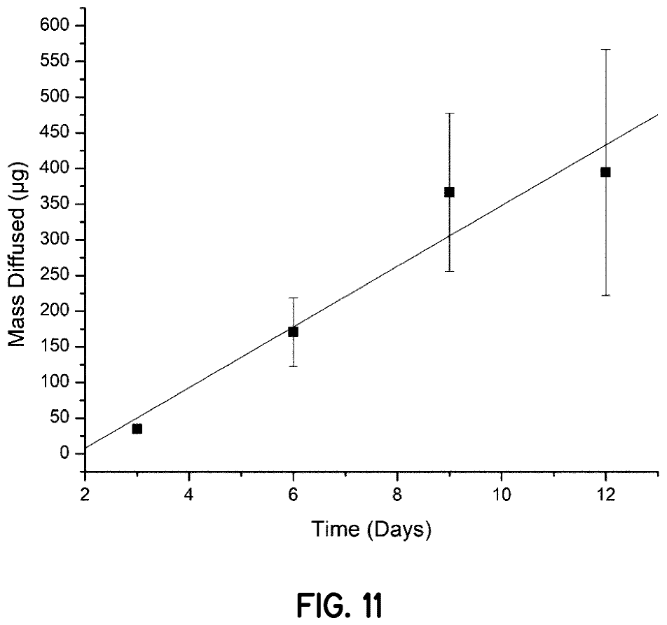

FIG. 11 shows the plot of Bev permeating across a 0.05 PEG PCL membrane. For the most part, the permeation is steady state allowing for the use of Equation 1 to determine the permeability coefficient from the slope of the line given by the permeation data. With a donor concentration of 50 mg/mL, an exposed membrane area of 0.78 cm², and 42.5 μg/day permeated across the membrane, the permeability was found to be 1.28E-8 cm/s. The partition coefficient determined that K=0.515 when using an equilibrium solution of 20 mg/mL.

Calculating the Stokes-Einstein diffusion coefficient and hindrance factor gives the final values necessary to use Equation 8 to calculate the effective diffusion coefficient. Using the accelerated degradation images to determine the change in pore size over time, the change in De over time can be incorporated into the modeling equation for a more accurate representation of the long-term release.

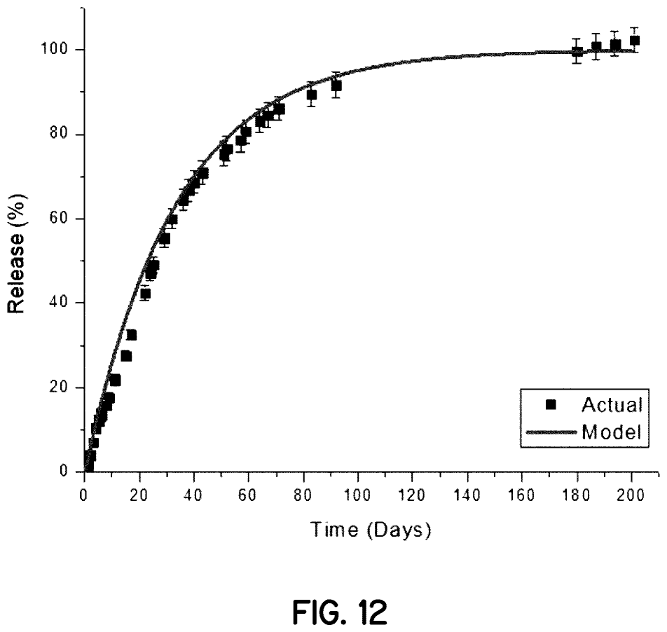

FIG. 12 shows the model created from the degradation data and compares it to the actual release data from a 1.25 mg Bev 0.05 PEG ratio implant in PBS. The model shows a faster release kinetics than the actual release data. The actual data begins to heavily deviate from the model around day 50 where the release begins to plateau compared to the model which begins its plateau around day 85. The model uses different De values corresponding to 0, 4, and 10 days of accelerated degradation. Using the time correlation found in FIG. 6, at 55 days, the De is from the 4 days degradation, and at 130 days, the 10 days degradation De was used. As the current model stands, the model predicts 99.56% release by 180 days.

Regarding the second experiment, permeability (P) and partition coefficient (K) of Bev were experimentally determined for parameters needed for the model fitting (Equation (4)). FIG. 11 shows the plot of Bev permeating across a 0.05 PEG PCL membrane. For the most part, the permeation is a steady state allowing for the use of Equation (2) to determine the permeability from the slope of the line given by the permeation data. With a donor concentration of 50 mg/mL, an exposed membrane area of 0.78 cm², and 42.5 μg/day permeated across the membrane, the permeability was found to be 1.28E−8 cm/s.

The partition coefficient determined experimentally was K=0.515 when an equilibrium solution of 20 mg/mL was used. The effective diffusion coefficient (Dₑ) was calculated based on Equation 9, calculating the Stokes-Einstein diffusion coefficient and hindrance factor. Table 5 summarizes the values used for the drug release kinetics model (Equation (4)) to generate FIG. 12.

TABLE 5
Values Used in Drug Release Kinetic Model

| Parameter | Value |
|---|---|
| Ri (cm) | 0.0413 |
| Ro (cm) | 0.0464 |
| L (cm) | 1 |
| K | 0.515 |
| Dₑ (cm²/day) | 5.59 × 10⁻⁶ |
| M_{∞} (mg) | 1.25 |

Drug Release Model: FIG. 12 shows the model fit with the release data from a 1.25 mg Bev 0.05 PEG ratio implant in PBS. As the current model stands, the model predicts a 99.56% release by 180 days compared to the 99.71% released in the actual data.

The equation predicted the accelerated drug release kinetics well, taking into account the total drug release, as shown in FIG. 12. Incorporating the time correlation from FIG. 4 instead of t and multiplying the result by a ratio of the final release percent for accelerated and natural conditions, we obtain a modified version of Equation (4):

$$\frac{{M}_{t}}{{M}_{∞}} = 1 - exp [ - \frac{( {R}_{i} L + {R}_{o} L + 2 {R}_{i} {R}_{o} ) {D}_{e} K \frac{t}{0.0807}}{{R}_{i}^{2} L ( {R}_{o} - {R}_{i} )} ] \qquad ( 13 )$$

Equation (13) can be used to model the accelerated release to compare how the model in natural conditions will represent the actual release in that condition.

### Discussion

After testing the No PEG, 0.05, and 0.1 PEG conditions, the data showed that increasing the PEG concentration in PCL increases the pore size, porosity, thickness of the membrane and the degradation rate. The increased porosity allowed for the natural or accelerated solution to penetrate into the membrane and increase the surface area for degradation and increasing the degradation rate. The decrease in the scale area during the beginning of degradation is most likely due to the breakdown of polymer chains on the surface. The initial degradation causes small channels to develop on the larger scales creating a larger number of smaller scales. As degradation continues the area around the small channels eventually levels out creating larger scales again while deepening the remaining ones. The decrease in pore size can be explained by new smaller pores developing during degradation and then merging to create larger pores which brings the pore size back up towards the end of the degradation period. The porosity held steady with the initial degradation because the new smaller pores did not cause a significant change in the porosity. It eventually decreased because the pore volume was decreasing and even though there may have been more total pores, the volume they took up was less than before degradation. The final increase in porosity can be attributed to the PCL between the pores degrading away and increasing the total volume the pores take up.

Correlating the amount of time in PBS and NaOH by the area under the curve for the release rates in both conditions demonstrated a linear relationship between the amount of time spent in PBS and the amount of time in 0.1 M NaOH. This linear relationship was verified by analyzing the change in area for the scale structures present on the implants during degradation. Comparing the 4 day with the 1- and 2-month scale sizes and the 10 day with the 4-month scale size verified the time comparisons made from the linear relationship. Using this relationship, accelerated condition pore diameter and pore size can be used to in the modeling equation to more accurately represent the release kinetics over time without the need of testing the release kinetics for 6 months in natural condition.

Regarding the second experiment, after testing the 0.0, 0.05, and 0.1 PEG conditions, the data showed that increasing the PEG concentration in PCL increases the pore size, porosity, thickness of the membrane, and the degradation rate. The increased porosity allowed for the natural or accelerated solution to penetrate into the membrane and increase the surface area for degradation and increase the degradation rate. The decrease in the scale area during the beginning of degradation is most likely due to the breakdown of polymer chains on the surface. The initial degradation causes small channels to develop on the larger scales creating a larger number of smaller scales. As degradation continues, the area around the small channels eventually levels out creating larger scales again while deepening the remaining ones.

The decrease in pore size can be explained by new smaller pores developing during degradation as well as swelling. The increase in pore size at the end of degradation can be explained by further degradation and merging of the smaller pores to create larger ones. The porosity held steady with the initial degradation because the new smaller pores formed were offset by swelling. It eventually decreased due to the swelling and decrease in pore size. The final increase in porosity can be attributed to the PCL between the pores degrading away to increase the total volume taken up by the pores.

Correlating the amount of time in PBS and NaOH by the ratio of natural and accelerated final releases, a linear relationship between the amount of time spent in PBS and the amount of time in 0.1 M NaOH can be determined. This linear relationship was verified by analyzing the change in area for the scale structures present on the implants during degradation. Comparing the 4 day with the 1 and 2 month scale sizes and the 10 day with the 4 month scale size verified the time comparisons made from the linear relationship. Using this relationship, the accelerated condition pore diameter and pore size can be used in the modeling equation to more accurately represent the release kinetics over time without the need for testing the release kinetics for 6 months in natural conditions.

The degradation kinetics in vivo looked similar to that in the physiological conditions in vitro, suggesting that the main degradation mechanism is chemical, not biological, specifically by OH—. Thus, it is expected that the 0.1 M NaOH condition would provide the accelerated condition for in vivo applications. However, biological degradation by the host's enzyme is pronounced when the molecular weight (Mn) reaches ˜5000 Da. Hence, further study may be necessary to determine the effect of the enzymes for the late stage beyond 6 months.

The natural condition model accurately predicts the long-term release of Bev from 0.05 PEG PCL implants. With a method of modeling release, any adjustments made to the implant such as adjusting the thickness, length, and PEG concentration can be plugged into Equation (4) to predict the long-term release kinetics. Using initial SEM data, permeability, and the partition coefficient, the long-term release can be modeled within a few weeks. Using this model, the daily dose can be calculated for varying amounts of loaded Bev and compared to current methods of care to better tailor the release and create effective treatment plans. The accelerated model can be used to compare the actual accelerated release and the theoretical release to determine how well the natural model will fit the release data. The accelerated methods used in this study can help expedite the development of biodegradable implants to treat various diseases using various types of mAbs other than Bev. The time correlation between the natural and accelerated degradation conditions (Equation (12)) is specific for the 0.05 PEG PCL capsule with the specific dimension, and the correlation can be different for other polymers with different morphologies, including porosities and thicknesses.

In contrast to alternative capsule implants, it was demonstrated that the drug release kinetics of monoclonal antibody release for over 6 months using a biodegradable polycaprolactone (PCL) capsule was not affected by degradation because of the slow degradation of PCL. However, as observed in the accelerated condition, degradation affecting the breakdown of the overall structure is expected beyond 12 months in physiological conditions. More importantly, it is fully understood the long-term drug release kinetics by fitting the first-order kinetics for a cylinder reservoir shape, implying that the kinetics can be tuned by model parameters, including dimensions of the capsule, and polymer materials. The model can also predict drug release kinetics in the accelerated degradation condition.

### EXAMPLES OF PCL, PLA, 90:10 PLGA, AND 50:50 PLGA CAPSULES

Dex release from four different types of polymers to confirm the feasibility of the mathematical models using permeability and partition coefficient for Dex release profile was studied. Particularly, capsules made from at least one of PCL, PLA, 90:10 PLGA, or 50:50 PLGA were studied. Dexamethasone sodium phosphate, a water-soluble salt form of Dex, was used. Due to the high water-solubility of dexamethasone sodium phosphate, it allows the administration of relatively high doses in a small volume of aqueous diluent for drug injection. The in vitro delivery efficiency for anti-inflammatory effects and the cytotoxicity of the implant were also tested using TNF-α stimulated human umbilical vein endothelial cell (HUVECs).

### MATERIALS AND METHODS

### Materials

Poly(caprolactone), PCL (MW 65,000-75,000), Poly(L-lactide), PLA (MW 100,000-125,000), Poly(lactide-co-glycolide), PLGA 90:10 (L:G 90:10, MW 100,000-200,000), Poly(lactide-co-glycolide), PLGA 50:50 (L:G 50:50, MW 15,000-25,000) were purchased from PolyScitech®, Inc (West Lafayette, IN). Dichloromethane (DCM), potassium chloride (KCl), and MTT (3-(4,5-Dimethylthiazol-2-yl)-2,5-Diphenyltetrazolium Bromide) were purchased from Fisher Chemical® (Waltham, MA). Dexamethasone sodium phosphate powder was purchased from The Lab Depot® (Dawsonville, GA). Human umbilical vein endothelial cell (HUVECs), EBMTM-2 Basal Medium and EGMTM-2 SingleQuots™ Supplements were purchased from Lonza® (Basel, Switzerland). Human IL-6 Matched Antibody Pair Kit and ELISA® Accessory Pack were purchased from Abcam® (Cambridge, UK). Corning UV-transparent microplate was purchased from Sigma Aldrich® (St. Louis, MO).

### Synthesis of Polymer Sheets

A polymer solution in DCM at 50mg/mL was transferred into a mold (1,500 μL) that was created by attaching two stainless-steel rectangles (4.9 cm×3.1 cm×1.5 cm, length×width×height) to a phone glass protector using superglue. DCM was slowly evaporated with a cover on at 15° C. overnight to create the polymer sheet. The dry sheet was then removed by a razor blade.

### Implant Fabrication

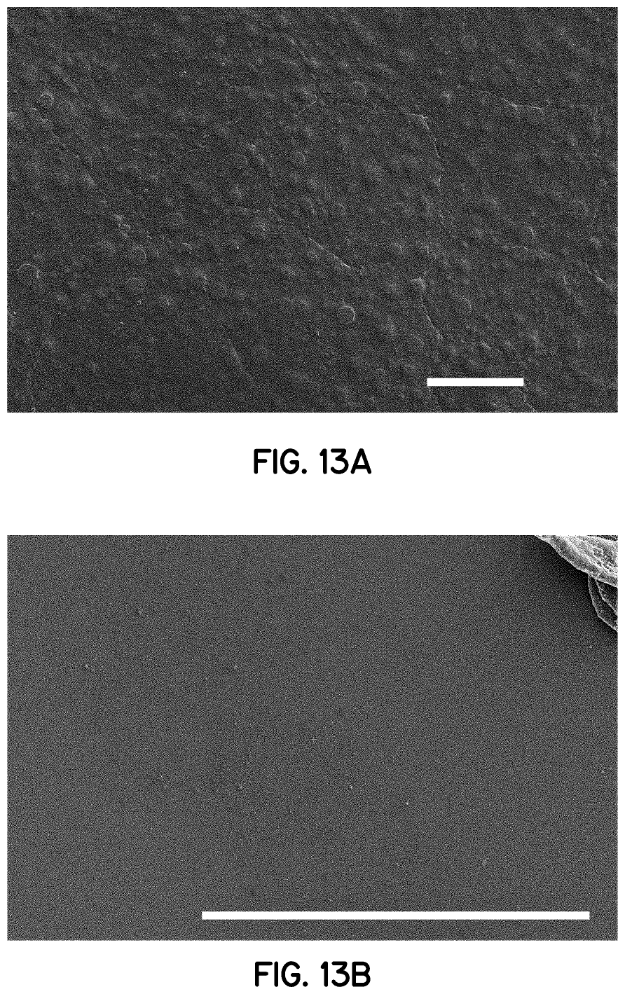

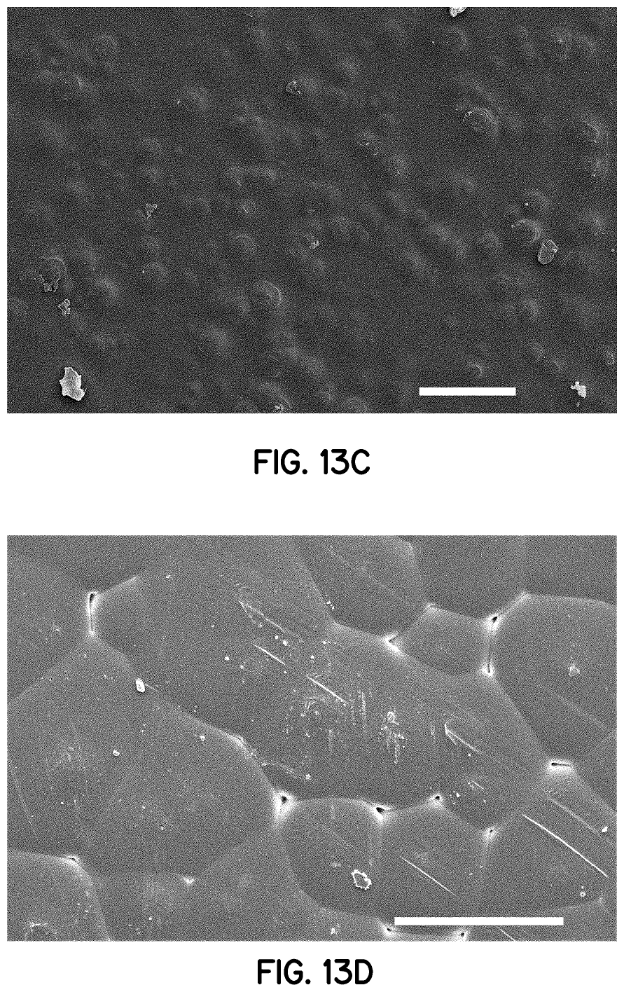

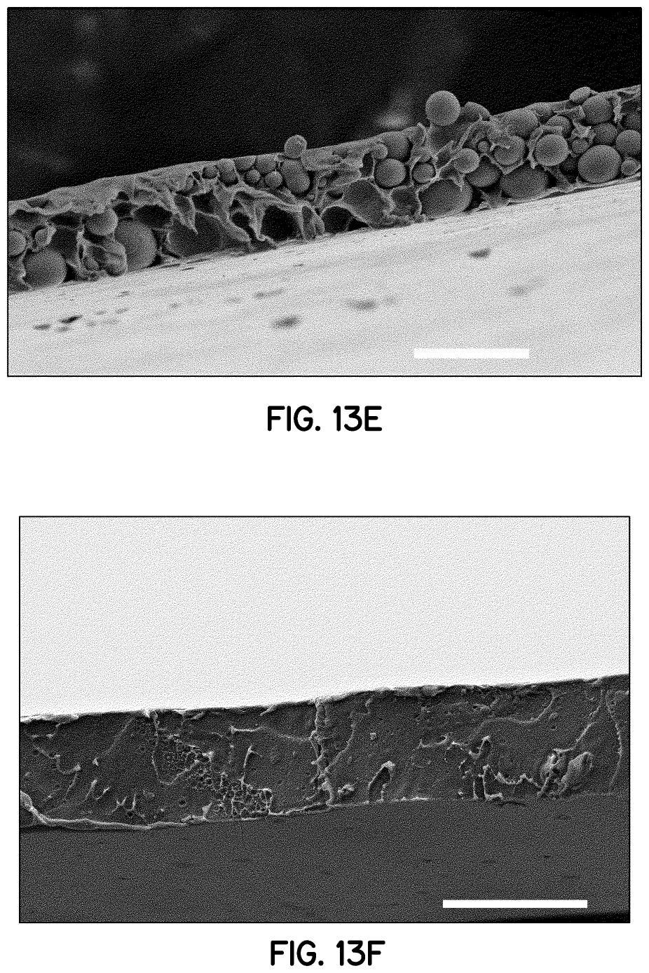

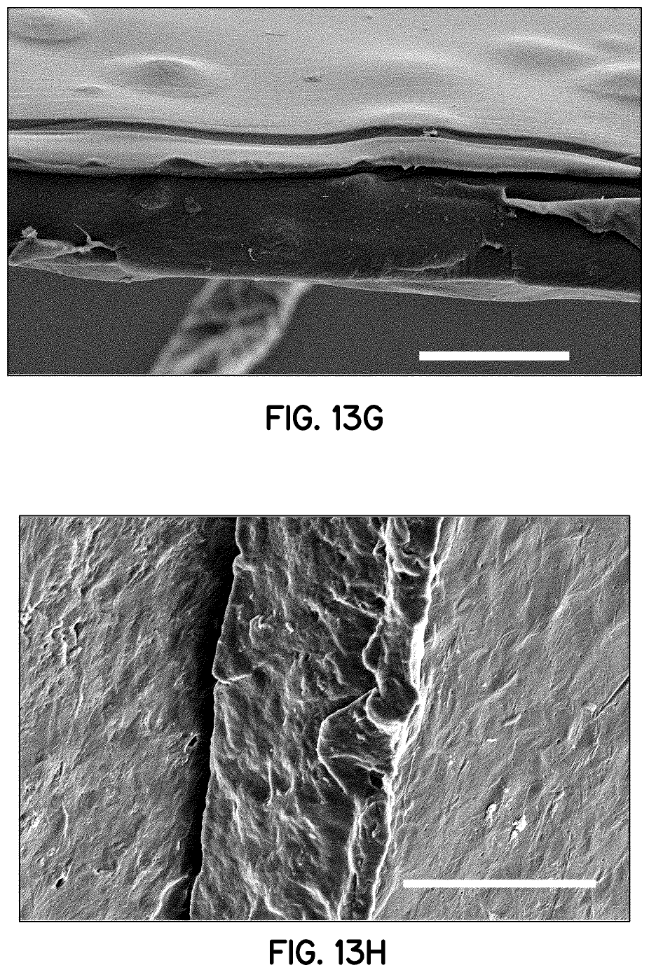

The polymer sheet was cut into 1 cm×0.5 cm pieces using a razor blade for implant fabrication. The pieces were then rolled around a 22-gauge needle at 45° C. to create a double layered tube with 1 cm in length and 0.0946 cm in diameter. Two implant tubes with 0.5 cm in length were created by cutting the 1 cm implant tube into half. One end of the tube was clamped with a 60° C. iron. A 26-gauge needle was used to load 1.3 μL of the 500 μg/μL Dex solution into the tube, resulting in 650 μg encapsulation. Once the Dex solution was loaded, the iron was used to seal the other end and then both ends were superglued to prevent any possible leakage. The implant fabrication process is shown in FIGS. 13A-H, and the dimension of the final implant is ˜0.9 mm in diameter and 5 mm in length. As shown in FIGS. 13A-H, the implant was placed inside an 18-gauge syringe needle with sealed ends. The final dimension of the implant is 5 mm in length and 0.464 mm in outer diameter. Specifically, FIG. 13A is an SEM image of a sheet of 50:50 PLGA. FIG. 13B is an SEM image of a sheet of 90:10 PLGA. FIG. 13C is an SEM image of a sheet of PLA. FIG. 13D is an SEM image of a sheet of PCL. FIG. 13E is a cross-sectional image of a sheet of 50:50 PLGA. FIG. 13F is a cross-sectional image of a sheet of 90:10 PLGA. FIG. 13G is a cross-sectional image of a sheet of PLA. FIG. 13H is a cross-sectional image of a sheet of PCL.

### Drug Release Profile

Implants loaded with the Dex solution were placed in a test tube with 1 mL of PBS and incubated at 37° C. For each polymer, four implants were made. The PBS solution was removed and replaced with fresh PBS every 24 h. The removed solution was then analyzed using UV-Vis at 240 nm to determine the Dex concentration. Using the concentration with calibration curve, the amount of Dex released was plotted to determine the release profile for a time period of 42 days.

### SEM Imaging

The surface and cross-section structures were analyzed using scanning electron microscopy (SEM) (Thermo Fisher Scios Dual Beam® SEM, Hillsboro, OR). The polymer sheets were attached to horizontal or vertical SEM sample holders using double sided graphite tape. The samples were sputter-coated with 5 nm platinum/gold nanoparticles for 10 s using Denton Vacuum Desk II® (Moorestown, NJ). 1 cm×0.5 cm sheets were coated in glycerol, submerged in liquid nitrogen, then fractured to obtain a clean cut to view the cross-section. Fractured sheets were then rinsed with DI water, dried overnight, and placed on vertical sample holders to image the cross-section. The thickness of the sheets was analyzed using ImageJ® (NIH).

### Porosity Measurement

The porosity of the polymer sheets was determined by using a setup of a humidity chamber with potassium chloride (KCl) as the saturated salt solution. A synthesized polymer sheet was cut into two 0.5 cm×0.5 cm pieces with a razor blade. A double layered polymer sheet was then created by placing the two pieces on top of each other and pressing them together at 45° C. Three double layered polymer sheets were made for triplicates. All dry weights of the double layered polymer sheets were recorded before taking inside the chamber. A weight boat filled with KCl was placed inside the petri dish. Once the double layered polymer sheets were moved into the humidity chamber, DI water was added to create a saturated KCl salt solution. The top of the petri dish was covered and wrapped with parafilm to prevent any possible leakage of the vapor. After 48 h, wet weights of the double layered polymer sheets were measured.

Porosity was calculated using:

$$ε = \frac{{V}_{w} - d}{{V}_{w}} * \frac{1}{RH} \qquad ( 12 )$$

where V_{d} is the volume calculated from the dry weight and the density of the polymer, V_{W} is the volume calculated from the wet weight considering the density of water and the polymer, and RH is the relative humidity of the saturated salt solution. In this study, the relative humidity of KCl, salt solution used, is 85% at 25° C.

### Permeability Measurement

Vertical Franz diffusion cells were used to conduct the permeability experiments. The donor side was filled with 200 μL of 30 mg/mL Dex solution and the receiver side was filled with 5 mL of PBS. The concentration of the donor solution was estimated based on the release profile. A 1.3 cm×1.3 cm double layered polymer sheet was used as the membrane. To measure the permeability, a 1.3 cm×1.3 cm double-layered polymer membrane was made in a similar way to the membranes in the porosity experiment. Once the membrane was securely placed between the donor and receiver chambers, 200 μL of the PBS solution was loaded for a one-hour equilibration. After equilibration, the PBS solution was exchanged by the donor solution and then the opening was sealed with para-film to reduce evaporation altering the concentration of the donor and receiver solutions. 300 μL of the receiver solution and 10 μL of the donor solution were removed every 24 h to determine the Dex concentration using UV-Vis. Fresh PBS was used to replace the removed receiver solution, and the donor side was also replaced by fresh donor solution. Depending on the remaining concentration of the Dex donor solution, fresh 30 mg/mL Dex solution might be used to replace the solution in the donor side.

After four days, the mass of Dex that permeated the membrane was plotted against time to get the release over time dQ/dt using Equation (2) described above:

$${C}_{i} \cdot P = \frac{1}{A} \cdot \frac{dQ}{dt} \qquad ( 2 )$$

where Cᵢ is the initial concentration of the Dex donor solution, P is the permeability coefficient of the membrane, Q is the amount of Dex permeated at time t, and A is the area of the exposed membrane.

### Partition Coefficient Measurement

Partition coefficient K was determined for the model equation used to predict the release kinetics. A 0.5 cm×0.5 cm double-layered polymer membrane was made in a similar way to the membranes in the porosity experiment to measure the partition coefficient. The membranes were soaked in 300 μL of PBS for 48 h. After 48 h the membranes were tipped on a Kimwipe to remove the excess surface liquid and then weighed to determine the wet mass of the membrane. The membranes were then dried overnight in a chemical hood. The dry membranes were then placed in 300 μL equilibrium Dex solution at a concentration of 11.95 mg/mL for 72 h for the membranes to absorb the Dex from the solution. After 72 h, the membranes were removed from the Dex solution. Kimwipe was used again to get rid of the excess solution on the membranes and then the membranes were placed in 300 μL of fresh PBS to extract Dex for 48 h. This extraction process was repeated until the amount extracted was at least 90% less than the first extraction based on the UV-Vis. The remaining equilibrium solution and all extraction solutions were analyzed using UV-Vis to determine the Dex concentration. The partition coefficient was then determined using the equation:

$$K = \frac{\frac{{M}_{e}}{W}}{\frac{{M}_{eq}}{V * ρ}} \qquad ( 13 )$$

where Mₑ is the total mass extracted, W is the wet mass of the membrane, M_{eq} is the amount of Dex left in the equilibrium solution, V is the volume of equilibrium solution left, and ρ is the density of the solution.

### Modeling Equations

The modeling equation used for predicting the release profile is

$$\frac{{M}_{t}}{{M}_{∞}} = 1 - exp [ - \frac{( {R}_{i} L + {R}_{o} L + 2 {R}_{i} {R}_{o} ) {D}_{e} Kt}{{R}_{i}^{2} L ( {R}_{o} - {R}_{i} )} ] \qquad ( 14 )$$

where Mₜ is the mass of Dex released at time t, M. is the mass of Dex loaded in the implant, Ri is the inner radius of the implant, Rₒ is outer radius of the implant, Dₑ is the effective diffusivity, K is the partition coefficient, and L is the length of the implant. This modeling equation follows Fick's law of diffusion of molecules from the lumen of a cylindrical implant through a membrane wall. Variables, Rₒ, Ri, and L, were measured from the implants directly. Partition coefficient K was determined experimentally as described herein.

Dₑ was determined by the following equation (Equation (9) above), assuming the Dex molecules diffuse through the pore spaces of porous media:

$$De = \frac{ε D}{τ} \cdot {K}_{r} \qquad ( 9 )$$

where ε is the porosity of the membrane, D is the Stokes-Einstein diffusion coefficient, τ is the tortuosity, and Kᵣ is the restrictive factor when the ratio of the molecule diameter (dₘ) and pore diameter (dₚ) is less than 1. The relationship between the diameters is expressed by (Equation (10) above):

$${K}_{r} = {[ 1 - \frac{{d}_{m}}{{d}_{p}} ]}^{4} \qquad ( 10 )$$

D, the Stokes-Einstein diffusion coefficient was calculated based on:

$$D = \frac{{k}_{B} T}{6 π η r} \qquad ( 15 )$$

where k_{B} is the Boltzmann constant, T is the temperature in Kelvin, η is the viscosity of water at 25° C., and r is the hydrodynamic radius of Dex. The porosity E was determined experimentally as described herein; the ratio of restrictive factor Kᵣ over tortuosity τ of all polymers tested was obtained by fitting the data. The release profile data were fit to Equation (14) using Excel®. The actual release at Day 5 and Day 14 was compared to the model prediction values and the percent error was calculated.

### Cell Culture and Enzyme-Linked Immunosorbent Assay (ELISA)

Human primary umbilical vein endothelial cells (HUVECs) were maintained in EBMTM-2 Basal Medium and EGMTM-2 SingleQuots™ Supplements (Lonza, Switzerland). HUVECs were grown in the cell incubator containing 5% CO2 at 37° C. When the cells were grown to 90% confluence, they were inoculated in 24-well plates. Briefly, 0.4 mL of HUVECs suspensions (8×104 HUVECs per well) were seeded in 24-well plates and incubated at 37° C, 5% CO2 for 24 h. Experimental conditions (PBS-implant, Dex-implant, and free Dex) were treated with the respective conditions at Dex concentration of 33 μM. After 24 h, all the conditions except the medium only were incubated with 20 ng/mL TNF-α for 24 h. The Dex concentration was selected based on the release kinetics; −33 μM of Dex was released from the 100 μg Dex-loaded implants within 24 h of incubation. At the end of experiments, the cell medium was collected and centrifuged at 2,000×g for 10 min to remove the particulate/debris materials. Then, supernatant was collected and stored at −20° C. until used. Each sample was pre-diluted using a standard dilution buffer to keep the sample concentrations within the range of standard curve. The concentration of pro-inflammatory cytokine (IL-6) in the cell culture supernatant was analyzed by the ELISA assay according to the manufacturer's recommendation (Invitrogen® human IL-6 ELISA kit). All tests were done in triplicate.

### Cell Viability Assay

The cell viability of HUVECs was determined using an MTT assay. The cytotoxicity of PBS-implant, Dex-implant and free Dex was investigated after 24 h of pretreating HUVECs followed by 20 ng/mL TNF-a treating for the other 24 h. Briefly, MTT reagent (5 mg/mL) in the medium was added to each 24 well plates after the supernatant were collected for the ELISA assay and incubated at 37° C. and 5% CO₂ in the cell incubator for 3 h. Then, the medium was removed and 200 μL of dimethyl sulfoxide (DMSO) was added to each well plate to dissolve the yellow formazan precipitates. After complete homogenization under shaking, 100 μL was collected from each well and added to 96 well plate and the absorbance was measured at 492 nm on a microplate spectrophotometer reader (Spectramax®, Molecular Devices, LLC). All experiments were done in triplicate.

### Results

### Characterization of the Polymer Sheets

The surface of 50:50 PLGA and PLA showed a round bumpy structure (FIGS. 13A and 13C). The 50:50 PLGA's round bumps range from 8-20 μm, while the PLA's bump size is consistently around 10-30 μm. 90:10 PLGA and PCL does not show the bumpy structure prominently (FIGS. 13B and 13D). All four polymers have a smooth bottom surface from being in contact with the mold during synthesis. The cross-section images of the 50:50 PLGA and PLA show a spherical structure (FIGS. 13E and 13G), which affects the bumpy structure on the surface in FIGS. 13A and 13C, respectively. 90:10 PLGA has small pores, between 0.5-1 μm, distributed throughout the cross-sections (FIG. 13F). PCL has a smooth cross-section (FIG. 13H). The spherical and bumpy structures in 50:50 PLGA and PLA are most likely caused by the hydrophilicity of the polymers during the evaporation of DCM. It is hypothesized that a relationship exists between the hydrophilicity of the polymer and the evaporation rate of DCM, such as the more hydrophilic the polymer, the faster the evaporation rate of DCM, causing the structures.

PCL has the thickest membrane with a thickness of 20.5±0.37 μm, followed by PLA with a thickness of 17.8±1.11 μm, 50:50 PLGA with 16.2±0.42 μm, and 90:10 PLGA with 16.3±0.42 μm. The thickness of the two PLGA ratios does not significantly differ, and they both are significantly different from PLA and PCL (p<0.05). PLA and PCL also has a significant difference in their thicknesses (p=0.025). The slight decrease in the thickness of the two PLGAs from the PLA may be due to the addition of glycolide. This addition reduces the number of branches from the lactide and reduces thickness slightly.

### Drug Release Profile From Polymer Capsules

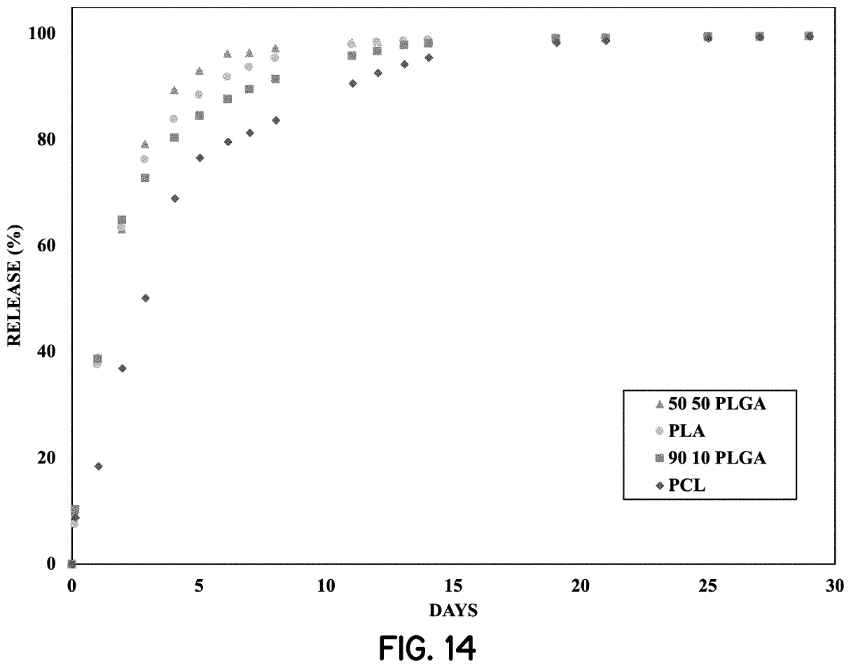

The release profiles from the implants show that almost 90% of Dex was released within ˜15 days for the four polymer implants as shown in FIG. 14. Four polymer implants' plateaued at ˜600 μg, with ˜50 μg of the Dex left in the polymer membrane. The ˜50 μg is left in the polymer tube dimension because of thermodynamic equilibrium, the partition coefficient. Using the partition coefficient value, average K=1.52, and Equation (13), Me interpreted as the amount of Dex in the polymer was calculated as 46 μg. Briefly,

$${M}_{e} = K ( \frac{{M}_{eq}}{V * ρ} ) W , where \frac{{M}_{eq}}{V * ρ}$$

was approximated as the solubility of Dex in water at 293.15 K0.02168 g/g, and W=0.5 cm×0.5 cm×40 pm×1.4 g/cm³. The release was fast in the first five days, releasing around 80% before slowing to release 20% in the next ten days. As the concentration gradient across the membrane decreases over time, the release rate with time decreases as expected. Based on the release profile, Dex releases slowest from PCL, fastest from 50:50 PLGA, probably due to the hydrophobicity, which will be discussed in the next section.

### Permeability (P), Partition Coefficient (K) and Porosity (ε) of the Polymer Sheets

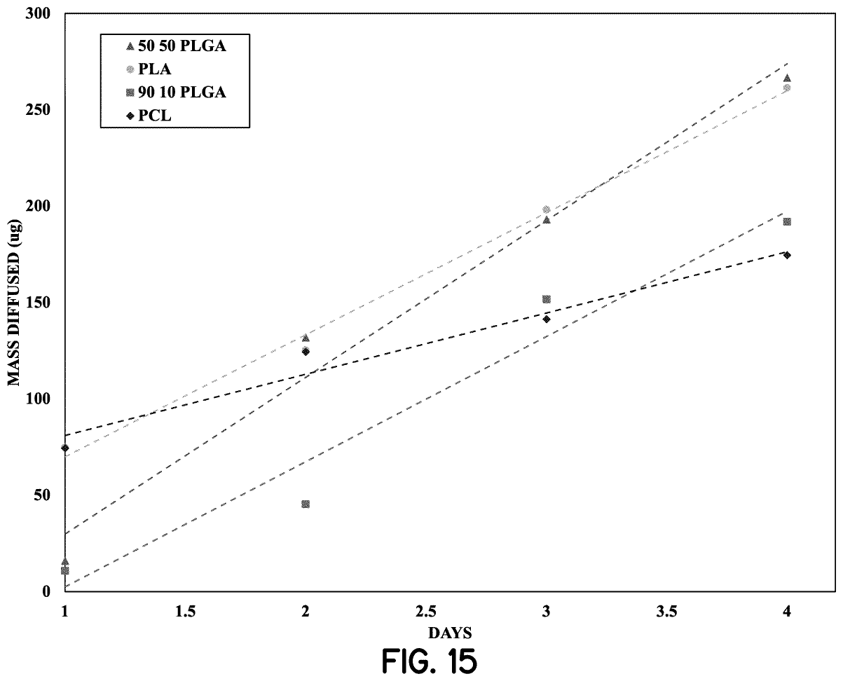

FIG. 15 shows the cumulative Dex amount that permeated across four different types of polymer membrane over time. The permeation is assumed at steady state and the permeability coefficient is determined from the slope of the line using Equation (2). Based on the slope of the lines, 31.751 μg/day, 63.299 μg/day, 64.96 μg/day, and 81.35 μg/day of Dex permeated across the membrane for PCL, PLA, 90:10 PLGA, and 50:50 PLGA, respectively. All permeability coefficients are listed in Table 6 for the four polymer sheets. For the partition coefficients, the concentration of equilibrium Dex solution was measured and determined using Equation (13) (Table 6). A trend between the partition coefficient and permeability coefficient is shown. As the partition coefficient increases, the permeability coefficient also increases, which is also directly related to the hydrophobicity of the polymers. Because Dex tested in this study is hydrophilic, the permeability through the hydrophobic materials is lower than the one through the hydrophilic materials.

The porosity values of the polymer sheets determined experimentally (Table 6) were similar. ANOVA tests were performed among the groups to determine if they are significantly different or not. All of the p-values were greater than 0.05, which means the porosity values among the samples were not statistically different.

TABLE 6
Experimental parameters: permeability coefficient, partition
coefficient, and porosity for all four polymers tested

| Polymer | Permeability | Partition | |
|---|---|---|---|
| sheets | coefficient P (nm/s) | coefficient K | Porosity (ε) |
| 50:50 PLGA | 0.4904 | 1.9519 | 0.0707 |
| PLA | 0.3816 | 1.6492 | 0.0759 |
| 90:10 PLGA | 0.3916 | 1.6019 | 0.0986 |
| PCL | 0.1915 | 0.8872 | 0.0577 |

### Drug Release Model

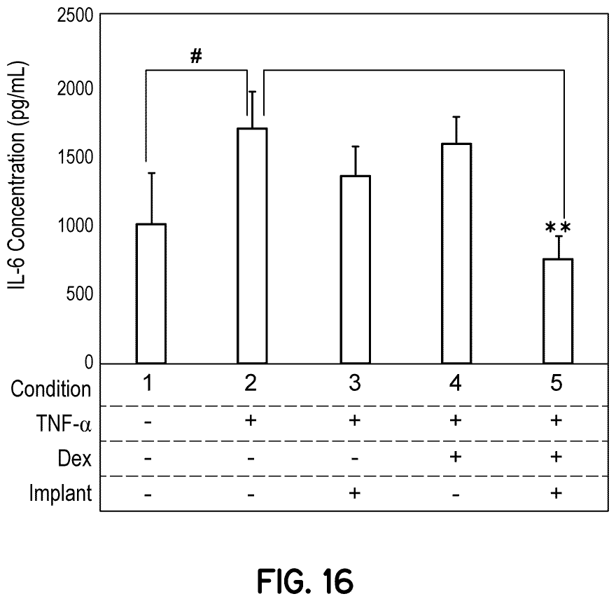

The Dex release profiles were fit to a cylinder reservoir shape model using Equation (14) for the four polymer implants as shown in FIG. 16. FIG. 16 is a bar graph showing IL-6-ELISA assay using TNF-α stimulated HUVECs—#p<0.05, and **p<0.01.The effective diffusivity was obtained by Equation (15) by adjusting the ratio of Kᵣ/τ in this modeling, as Kᵣ and τ both depend on the pore diameter, and this will be further addressed in the discussion section. In our case, SEM was not capable of capturing the pore diameter for the polymers. By adjusting Kᵣ/τ and using the experimentally determined values including permeability coefficient and porosity, the models fitted the actual release data relatively well in all four implants. All the values used in the modeling are shown in Tables 5 and 6. At day 5, the actual Dex release for PCL, PLA, 90:10 PLGA, and 50:50 PLGA was 67.42% (438.23 μg), 81.63% (530.60 μg), 78.03% (507.20 μg), and 85.85% (558.03 μg), respectively, and the model's calculated release was 65.66% (426.79 μg), 84.31% (548.02 μg), 83.61% (543.47 μg), and 87.87% (571.16 μg), respectively. The percent error between the actual and model release was 2.61%, 3.28%, 7.15%, and 2.35%, respectively. At day 14, the actual Dex release for PCL, PLA, 90:10 PLGA, and 50:50 PLGA was 86.95% (565.18 μg), 91.27% (593.26 μg), 90.59% (588.84 μg), and 91.02% (591.63 μg), respectively, and the model's calculated release was 89.46% (581.49 μg), 92.21% (599.37 μg), 92.18% (599.17 μg), and 92.30% (599.95 μg), respectively. The percent error between the actual and model release was 2.88%, 1.03%, 1.76%, and 1.41%, respectively. The overall trend for the release profile matched for all the polymer implants, indicating that the Dex release profile from the capsules followed the first order kinetics of a cylindrical reservoir. The Dex release from the PCL was the slowest, implying that the Dex PCL implant would be ideal for a sustained-release over 21 days among the four polymers tested.

TABLE 7
Fitting parameters for Kᵣ/τ and Dₑ from Equation (15)

| Polymer | | | | | Dₑ |
|---|---|---|---|---|---|
| capsules | Ri (cm) | Rₒ (cm) | L (cm) | Kᵣ/τ | (cm2/day) |
| 50:50 PLGA | 0.0413 | 0.0464 | 0.5 | 0.001059 | 2.84 × 10⁻⁵ |
| PLA | | | | 0.000942 | 2.71 × 10⁻⁵ |
| 90:10 PLGA | | | | 0.000721 | 2.69 × 10⁻⁵ |
| PCL | | | | 0.001169 | 2.55 × 10⁻⁵ |

### Dex-implant Reduces TNF-α-Induced Inflammation

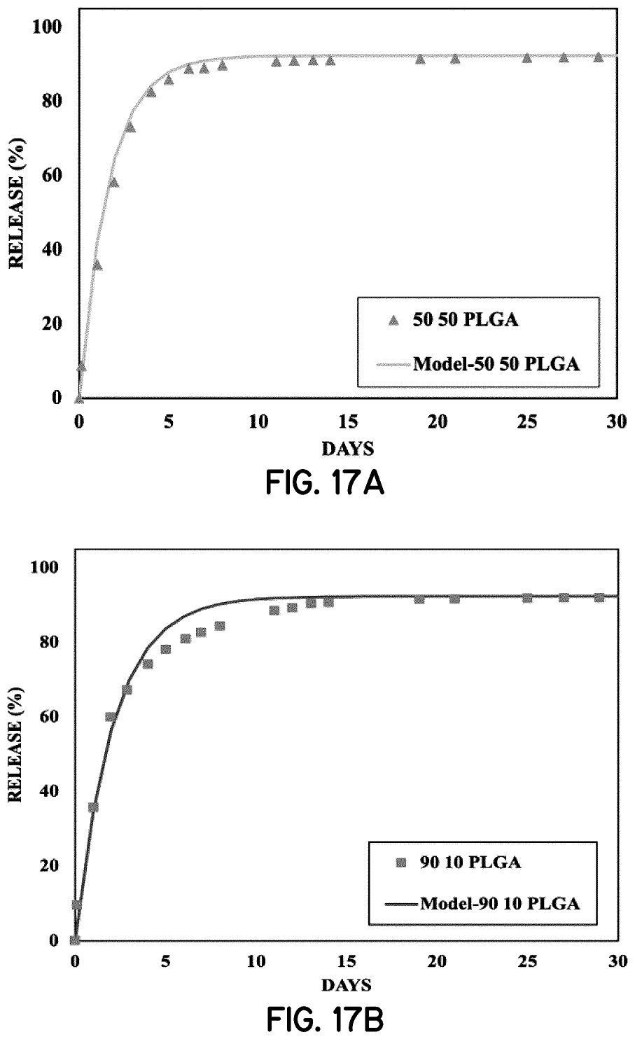

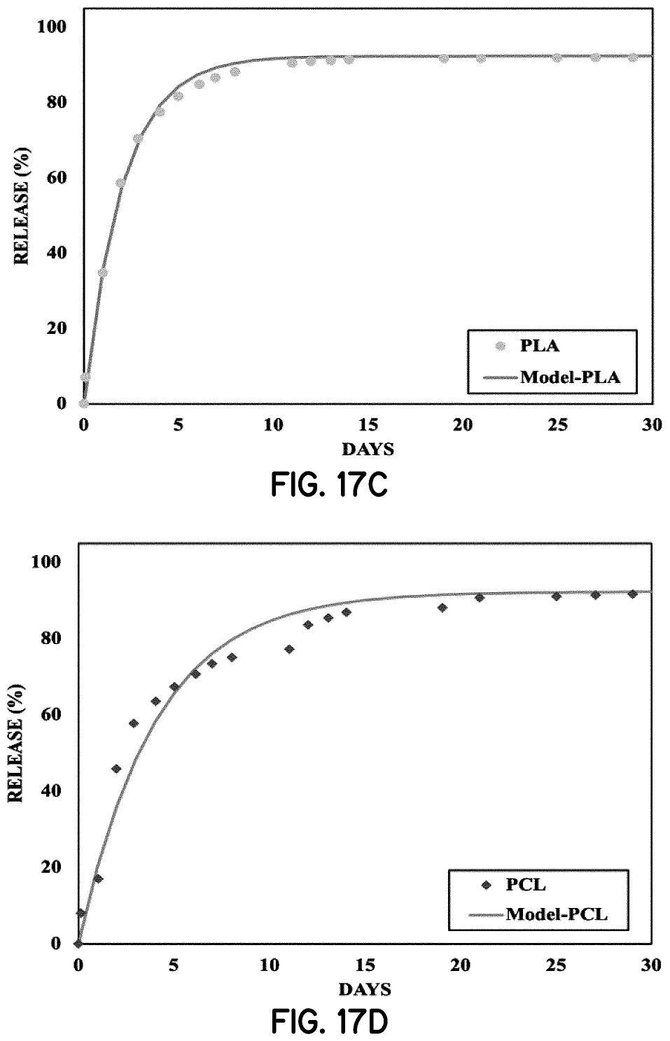

In a preferred embodiment, the PCL showed the most sustained- release, and may be selected to test the drug delivery efficiency in vitro and the cytotoxicity. To investigate therapeutic effects of the Dex-loaded PCL implant (condition 5) to protect HUVECs from TNF-α-induced inflammation, the levels of pro-inflammatory cytokine, IL-6, were quantified using ELISA assays. As seen in FIGS. 17A-D, the ELISA results show that the concentration of IL-6 was significantly increased (#p<0.05) in the TNF-α stimulated HUVECs (condition 2) in comparison to the negative controls (treated only with medium, condition 1), which confirms TNF-α induces HUVECs inflammation. Specifically, FIG. 17A is a graph of a model fitting for 50:50 PLGA to measured data. FIG. 17B is a graph of a model fitting for PLA to measured data. FIG. 17C is a graph of a model fitting for 90:10 PLGA to measured data. FIG. 17D is a graph of a model fitting for PCL to measured data. The IL-6 level in the cell culture medium significantly decreased (**p<0.01) in the Dex-loaded implants (condition 5) compared to condition 2, indicating the Dex was effectively delivered to the HUVECs to inhibit inflammation. Interestingly, the IL-6 level in condition 4 with the free Dex (condition 4) was not significantly decreased, which may imply that sustained slow release of Dex from implant for 48 h is more effective than single time injection of free Dex to reduce TNF-α-induced HUVECs inflammation. Lastly, the IL-6 level of the PBS-implant (condition 3) was not statistically different from the positive control (condition 2), indicative of no effect of the implant only on the inflammation inhibition.

### Effects of PBS-implant, free Dex and Dex-loaded Implant on HUVECs Viability

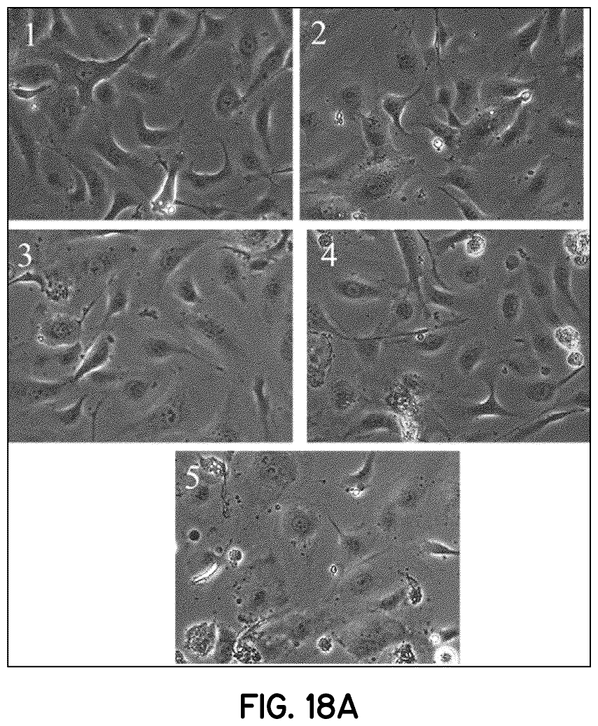

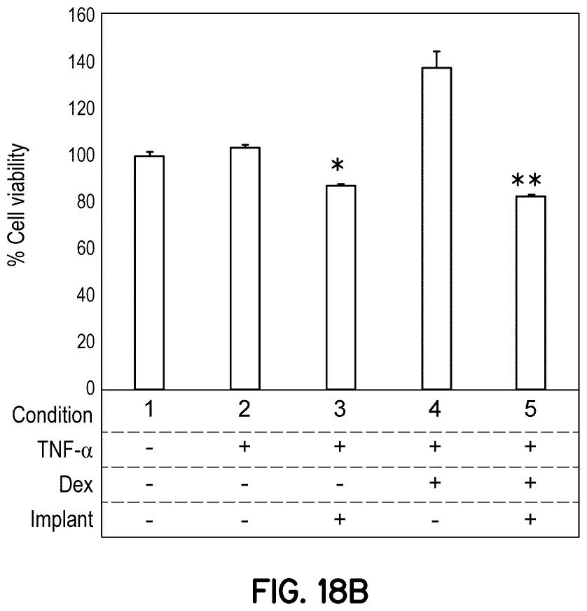

The cytotoxicity effects of PBS-implant (condition 3), free Dex (condition 4) and Dex-loaded implant (condition 4) were investigated via optical imaging and MTT assay using TNF-α stimulated HUVECs. As shown in FIG. 18A, more than 80% of HUVECs were alive in all treatment groups, including free Dex, PBS-implant, and Dex-loaded implant, indicating that the Dex with the dose tested and the polymer implant have negligible toxicity to HUVECs. Although the cells in all conditions looked healthy in the microscope images, the MTT assay results show that the PBS implant and Dex-loaded implant (conditions 3 and 5, respectively) had significantly lower cell viability than the negative control (condition 1) with p-values 0.02 and 0.005, respectively.

### Discussion

The release profile in FIG. 14 shows that almost 100% Dex was released from the hydrophilic implants in the first 15 days, which followed the first-order release kinetics. The release from the PCL capsule was slow at the beginning compared to the other three polymers, whereas the other three polymers had similar release for the first few days. The difference in the release appeared at day 5, in which the 50:50 PLGA released fastest compared to the other three.

Dexamethasone sodium phosphate is a hydrophilic drug. Theoretically, as the hydrophobicity of the polymer sheet increases, the slower the Dex releases from the capsules. PCL is a hydrophobic polyester used in many applications. PLGA is a copolymer of poly(lactic acid) (PLA) and poly(glycolic acid) (PGA). Because the methyl side groups in PLA makes it more hydrophobic than PGA, 90:10 PLGA is more hydrophobic than 50:50 PLGA. PCL is the most hydrophobic, and 50:50 PLGA is the most hydrophilic out of the four polymers, which is attributed to the release kinetics.

The permeability coefficient is a quantitative measure of the rate at which molecules can cross a membrane. A high permeability coefficient indicates the rate of the flow is high, which is directly related to the effective diffusion coefficient Dₑ. The trend of the permeability exactly matched the values of Dₑ (Table 7). Although Dₑ described in Equation (15) is a function of physical properties of the porous media, such as porosity and tortuosity, our results suggest that the Dex diffusion through the media is not solely governed by the physical properties but also the chemical properties of the membrane. This is because the Dₑ did not exactly follow the trend porosity ε, but followed the partition coefficient. The partition coefficient is a measure of a solute's solubility or distribution in the media. It was discovered, the greater the partition coefficient, the higher the permeability of the membrane to the solute. 50:50 PLGA has the greatest partition coefficient from experiments, and the permeability of Dex through the materials was the greatest. Thus, the results suggest that Dex diffusion through the polymer capsule is not mainly through the pores of the capsule membrane, but through the dissolution in the membrane.

In the modeling, because Kᵣ and τ were not experimentally determined, the ratio of Kᵣ/τ was fit to the actual release data. Many studies have made an effort to establish the relationship tortuosity, τ, and porosity, ε. ε is a function of void volume over total volume. The void volume can be estimated using the volume of single pore times the number of pores inside the members. By assuming the shape of the pores as spheres, the volume of a single pore is 4/3 πrₚ, where rₚ is the radius of the pore. Thus, porosity is proportional to rₚ³. On the other hand, Kᵣ is proportional

$${[ 1 - \frac{{d}_{m}}{{ε}^{- 1 / 3}} ]}^{4}$$

by substituting the relationship between pore diameter and porosity into the equation. This derivation indicates that as porosity increases, the ratio of Kᵣ/τ decreases. Porosity is inversely proportional to the ratio of Kᵣ/τ. In our study, PCL, with the smallest porosity measured as 0.0577, has the largest Kᵣ/τ ratio of 0.001169, 90:10 PLGA with the largest porosity measured as 0.0986 having the smallest Kᵣ/τ ratio of 0.000721, and the other two polymers have very similar porosity and Kᵣ/τ ratio, which follows the derivation.

In addition to physiochemical characterization of polymer implant, cytotoxicity and biological application using in vitro cell tests were also explored. HUVEC stimulated with TNF-α produces proinflammatory mediators, which will aggravate endothelial dysfunction. Our experimental findings show that Dex or Dex-loaded implant reduces HUVECs inflammation induced by TNF-α by decreasing the expression of pro-inflammatory cytokines, IL-6. Most interestingly, the IL-6 concentration in the cell culture medium significantly decreased in the HUVECs treated with Dex-loaded implant than the free Dex, which is probably due to the sustained release from the implant over time that can persistently inhibit IL-6 expression. Hence, the dosing method is highly important for effective Dex delivery to inhibit the expression of pro-inflammatory cytokines, which in turn enhances its anti-inflammatory effects. Although statistically not significant (p=0.095), the IL-6 level from the negative control (condition 1) is lower than condition 5. This may be due to the lower cell viability level of condition 5 than condition 1, as shown in FIG. 18B. Similarly, the reason why the IL-6 level from the PBS-implant (condition 3) is less than condition 2, although statistically not significant (p=0.078) is also probably because of the lower cell viability of condition 3 compared to condition 2. Interestingly, HUVEC treated with free Dex (condition 4) showed the highest cell viability and IL-6 level. It is believes this is because the cell was treated with Dex prior to the TNF-α administration. Some literature shows that Dex increases HUVEC proliferation in the presence of VEGF and pro-inflammatory responses when Dex is administered prior to TNF-α.

Finally, the MTT viability assay showed that more than 80% of HUVECs were alive in all treatment groups, indicating that synthesized polymer implant or Dex at tested concentration has minimal toxicity to HUVECs.

It was demonstrated that the release trend of Dex inside the polymer tubes is dependent on the hydrophobicity of the drug and the polymer, in which PCL releases Dex in the form of DSP slowest. Furthermore, by testing the permeability and partition coefficient of each polymer, the release kinetics of different polymers can be predicted using first-order kinetics modeling for a cylinder reservoir shape with known dimensions of the capsules. Finally, our cell test proved that the PCL Dex implant decreases IL-6 concentration more compared to the free Dex due to a sustained release over 48 h with minimum cell viability.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

1. A biodegradable capsule implant comprising:
a capsule body, comprising a polymer, having an inner surface and an outer surface, the inner surface defining a hollow interior space;
a therapeutic agent housed in the hollow interior space; and
a plurality of pores on the capsule body,
wherein the capsule body is configured to absorb the therapeutic agent at the inner surface and desorb the therapeutic agent out of the outer surface.

2. The biodegradable capsule implant of claim 1, wherein the polymer comprises an aliphatic polyester selected from a group consisting of polycaprolactone (PCL), poly(lactic acid) (PLA), 90:10 poly(lactic-co-glycolide) (PLGA), and 50:50 PLGA, and combinations thereof.

3. The biodegradable capsule implant of claim 1, wherein the polymer comprises polycaprolactone (PCL).

4. The biodegradable capsule implant of claim 1, wherein the therapeutic agent comprises either an antibody or an anti-inflammatory agent.

5. The biodegradable capsule implant of claim 4, wherein the therapeutic agent comprises the antibody.

6. The biodegradable capsule implant of claim 5, wherein the antibody comprises Bevacizumab or ranibizumab.

7. The biodegradable capsule implant of claim 4, wherein the therapeutic agent comprises the anti-inflammatory agent.

8. The biodegradable capsule implant of claim 7, wherein the anti-inflammatory agent comprises dexamethasone.

9. The biodegradable capsule implant of claim 7, wherein the anti-inflammatory agent has a therapeutic effect when treating tumor necrosis factor alpha-induced inflammation.

10. (canceled)

11. The biodegradable capsule implant of claim 1, wherein each of the plurality of pores has a diameter less than 1 micron in length.

12. The biodegradable capsule implant of claim 1, wherein the capsule body further comprises polyethylene glycol.

13. The biodegradable capsule implant of claim 12, wherein the capsule body comprises the polyethylene glycol and the polymer in a weight ratio of between more than 0.0 and less than or equal to 0.1 of the polyethylene glycol to the polymer.

14. The biodegradable capsule implant of claim 1, wherein the therapeutic agent is a first therapeutic agent, and the biodegradable capsule implant further comprises a second therapeutic agent.

15. The biodegradable capsule implant of claim 14, wherein the first therapeutic agent is Bevacizumab and the second therapeutic agent is ranibizumab.

16. A method of supplying a therapeutic agent to a recipient, the method comprising:
providing a biodegradable capsule implant comprising:
a capsule body, comprising a polymer, having an inner surface and an outer surface, the inner surface defining a hollow interior space;
a therapeutic agent housed in the hollow interior space; and
a plurality of pores on the capsule body,
wherein the capsule body is configured to absorb the therapeutic agent at the inner surface and desorb the therapeutic agent out of the outer surface,
wherein the biodegradable capsule is configured to release the therapeutic agent for a predetermined amount of time when the biodegradable capsule implant is administered to the recipient.

17. The method of claim 16, wherein the predetermined amount of time is at least 50 days.

18. The method of claim 16, wherein the predetermined amount of time is at least 200 days.

19. A method of making a biodegradable capsule implant, the method comprising:
stacking a plurality of polymer sheets onto each other;
rolling the plurality of polymer sheets, thus stacked, circumferentially to form a generally cylindrical shape defining a hollow cylindrical space, the hollow cylindrical space being configured to receive a therapeutic agent therein, the generally cylindrical shape comprising a first open end and a second open end;
submerging the generally cylindrical shape into a deionized water bath to form a plurality of pores through the generally cylindrical shape.
depositing the therapeutic agent in the generally cylindrical space via either the first open end or the second open end;
sealing the first open end and the second open end to form the biodegradable capsule implant.

20. A method of making a biodegradable capsule implant, the method comprising:
stacking a plurality of polymer sheets onto each other;
rolling the plurality of polymer sheets, thus stacked, circumferentially to form a generally cylindrical shape defining a hollow cylindrical space, the hollow cylindrical space being configured to receive a therapeutic agent therein, the generally cylindrical shape comprising a first open end and a second open end;
sonicating the generally cylindrical shape to form a plurality of pores through the generally cylindrical shape;
depositing the therapeutic agent in the generally cylindrical space via either the first open end or the second open end;
sealing the first open end and the second open end to form the biodegradable capsule implant.
